(19) United States
(12) Patent Application Publication
(10) Pub. No.: US 2006/0105013 A1
(43) Pub. Date: May 18, 2006

(54) Osteopontin-coated surfaces and methods of use

(76) Inventors: Samy Ashkar, Boston, MA (US); Jairo Salcedo, Boston, MA (US)

Correspondence Address: PATREA L. PABST;PABST PATENT GROUP LLP, 400 COLONY SQUARE, SUITE 1200, ATLANTA, GA, 30361, US

(21) Appl. No.: 09/981,845

(22) Filed: Oct. 18, 2001

Related U.S. Application Data
(60) Provisional application No. 60/327,273, filed on Oct. 5, 2001.
(60) Provisional application No. 60/241,248, filed on Oct. 18, 2000.

(51) Int. Cl. A61K 38/17; C07K 14/435

(52) U.S. Cl. 424/423; 530/350; 514/8; 514/12

(57) ABSTRACT

An osteopontin containing implant which increases the rate of osseointegration and the percentage of bone apposition is described. In one embodiment, the implant includes osteopontin or an active fragment thereof or an active peptide derived thereform. In another embodiment, the implant includes a material suitable for use in vivo within a subject in combination with a releasable form of osteopontin forming an osteopontin containing implant. The disclosed osteopontin derived peptides bind to various cell types and play important roles in cellular differentiation and/or motility. Many of these interactions are mediated by integrins as disclosed. Antibodies provide a mechanism to abolish or attenuate the activities of the claimed peptides.

This application is related to U.S. Provisional Application No. 60/327,273 filed on Oct. 5, 2001, U.S. Provisional Application No. 60/241,248 filed on Oct. 18, 2000.

### BACKGROUND OF THE INVENTION

The process that leads to successful osseointegration of an implant into the surrounding tissues is a complex one that involves cell migration, attachment, differentiation, proliferation, extracellular matrix synthesis and finally mineralization of that matrix. Implant materials are as biocompatible as their surface chemistry allows for a favorable interaction with the biological molecules relevant for that tissue.

For example, placement of endosseous dental implants has been limited to areas of favorable bone character, and fixtures must remain unloaded after placement for considerable periods of time. The primary challenges faced in the fabrication of new endosseous implants are to increase the rate of osseointegration and the percentage of bone apposition. Histological analysis of integrated titanium (Ti) implants into bone tissue revealed that many clinically successful implants are 30-60% apposed directly by mineralized bone. The rest of the implant surface has been found to be apposed by fibrous tissue and unmineralized collagen fibers. It is desirable that the entire circumference of the osseointegrated implant be directly apposed by mineralized bone tissue.

Extracellular matrix proteins, especially certain adhesion molecules, play a role in bone repair and morphogenesis. These molecules can modulate gene expression through cell surface-extracellular matrix interactions. The interaction between the titanium oxide layer of dental implants and certain extracellular matrix proteins may be a prerequisite for reproducible direct apposition of bone to titanium implants.

The adherence of cells to extracellular matrices (ECM) is a receptor mediated event leading to the assembly and reorganization of specific extracellular, transmembrane, and cytosolic components. Integrins represent a superfamily of cell surface proteins, most of which are composed of heterodimers of an α-subunit and a β-subunit. Examples of receptors and integrin type receptors are fibronectin and vitronectin receptors of fibroblasts, IIb/IIIa surface glycoproteins, and those discussed below. Cell surface receptors belonging to the integrin superfamily are recognized as critical players in the adhesion to the ECM and are intermediate messengers, relaying the recognition signal to cellular events such as motility, secretion, gene expression, differentiation, and contact or anchorage-dependent growth. Therefore, proteins or compounds that bind to these described receptors may influence a wide range of biological processes.

Osteopontin (OPN) and many peptides derived therefrom are such proteins. While aspartate residues in osteopontin appear to by critical for hydroxyapatite binding, the RGD sequence appears to mediate cell attachment via integrin receptors and thereby activate signal transduction pathways with the cell. Cleavage of osteopontin by thrombin has been reported to enhance the ability of cells to attach and spread in vitro (Senger et al., 1994, Mol. Biol. Cell., 5, 565-574), suggesting that thrombin cleavage makes the RGD motif more accessible. OPN has been localized to both bone and cementum and is the only protein detected, thus far, at the interface between bone (or cementum) and implants. Furthermore, osteopontin has also been detected within cementum at the site of insertion of Sharpeys fibers into cementum, suggesting a structural role of the protein in periodontal ligament formation. Sharpey's fibers are collagenous fibers that extend from periodontal ligaments into bone tissue and thereby provide stable connections with teeth, for example. Several laboratories have demonstrated differential attachment of osteoclasts, osteoblasts, macrophages or endothelial cells to surfaces coated with a variety of osteopontins. The “cryptic” nature of osteopontin is also exploited to investigate potential avenues for cell binding and influencing cellular differentiation and migration.

Matrix and Environment Dependent Differentiation

Attachment and proliferation of cells to a provisional matrix does not guarantee that these cells will differentiate into mature cells and express the necessary genes for proper matrix synthesis. When cells initially encounter a bio-matrix or extracellular matrix they will either attach and spread or they will undergo apoptosis. If the cells attach and spread, the cells are faced with two further alternatives, entering into the G1 phase of the cell cycle and proliferate, or up-regulating the expression of certain differentiation genes. Which alternative the cell “chooses” may depend on factors such as local extracellular signals and/or cues, and the type of cell (for example, whether or not the cell is terminally differentiated, precursor, or stem). Increasing evidence demonstrates that contact with the appropriate extracellular matrix is required to suppress the potential for cells to trans-differentiate. Trans-differentiation is defined as the process whereby a differentiated cell begins to express genes associated with another phenotype; for example, when epithelium cells become mesenchymal.

One of skill in the art will appreciate the complexity of the pathways that lead to committed precursor cells and differentiated cells. Differentiated cells originate from “primitive” cells, called stem cells. Generally, the stem cell is pluripotent and divides to either generate more pluripotent stem cells or committed precursor cells. Committed precursor cells are irreversibly determined to produce only one or a few types of cells. These cells also divide very rapidly but only for a limited number of times. After a series of rapid cell divisions, they develop into differentiated cells, wherein a contribution is made to the surrounding matrix. Driving this process of cellular development is motility (chemotaxis and/or haptotaxis) and/or proliferation, wherein motility and proliferation are regulated by, for example, increasing or decreasing gradients of, for example, peptides, proteins, cytokines, nutrients and/or hormones which bind to receptors on the cell surface.

Human osteoblast cell lines undergo a coordinated temporal expression of osteoblast phenotypic markers during their differentiation in vitro and produce a mineralized extracellular matrix. This bone developmental system is ideal for studying the interaction between titanium surfaces and bone cells in vitro.

Peptides that exhibit activity similar to OPN, and novel peptides that interact with many receptors including those, for example, αᵥβ₃, αᵥβ₅, 4β₁, 2β₁, VCAM, ICAM, CD44, V₃Vₓ have been identified. These receptors are found on the surfaces of various cell types, including those found on stem, limited potential precursors, precursors, committed precursors, and differentiated cells, and provide internal signals based upon the extracellular cue(s) they recognize. Integrin-type cell receptors mediate the adhesion of the cell to the extracellular surroundings or matrix. These receptors also govern molecular signal transduction events and pathways inside of the cell in response to whatever compound, peptide, protein or extracellular cue it recognizes and binds. Many cell types require anchorage to a matrix to ensure growth and viability and many integrins have provided this link between the extracellular matrix and essential internal cellular activity. Antibodies directed towards any of the above identified receptors may be used in attenuating or completely abolishing the activity of interacting peptides.

The peptides function in bringing stem cells, precursor cells (committed and uncommitted), and differentiated cells into contact with bone, cementum, PDL matrices or other biomaterials. Obtaining compounds that exert their activity by binding to the receptors, such as αᵥβ₃, αᵥβ₅, 4β₁, 2β₁, VCAM, ICAM, CD44, V₃Vₓ, allows one to influence or dictate differentiation process and recruitment. Once recruited to the matrix of interest, cell-cell communication increases the overall effectiveness of the compound.

Cell Attachment and Recruitment in Disease

Eosinophils, which make up one to three percent of the total white blood cell count, have been shown to contribute to a variety of diseases. Chronic allergic diseases such as bronchial asthma, or syndromes such as eosinophilic fasciitis or eosinophilic gastroenteritis are characterized by preferential accumulation of eosinophils at sites in airway inflammation in asthma, infiltration of eosinophils in affected muscle, tissue and fascia, and eosinophilic infiltration of mucosa, submucosa and muscularis of the small bowel, respectively. Eosinophils interact with ligands, the extracellular matrix, endothelial cells and epithelial cells via many of the same cell adhesion receptors and integrins discussed above. These interactions are critical to the infiltration and accumulation of eosinophils. Mechanisms by which one can control the migration process of cells like eosinophils, will be useful in the treatment of debilitating diseases. Integrins and integrin-like receptors therefore play a critical role in the unwanted accumulation of many cell types in human disease.

### SUMMARY OF THE INVENTION

Molecules and/or compounds involved in directing the migration or the attachment of cells to matrices of a particular environment are of interest in the presently described invention. The implants coated with these these peptides increase the rate of osseointegration and the percentage of bone apposition. Implant surfaces should have such properties which permit the phenomenology of the relevant cells. The achievement of reproducible biological integration of implants calls for a delineation of the molecular biological events relevant to the morphogenesis of the desired interfacial tissue. Material surfaces that can not bind the macromolecules supportive of osteoblast function, are not likely to make a good bone implant. An enhanced rate of osseointegration and/or augmented percentage of bone apposition around implants or cell recruitment systems of the invention increases implant placement indications, expedites loading time, and opens new fields for research in implant materials.

In some embodiments, the implant includes a material suitable for use in vivo within a subject in combination with a releasable form of osteopontin forming an osteopontin containing implant.

In another embodiment, the implant includes a material suitable for use in vivo within a subject in combination with at least two osteopontin polypeptides forming an osteopontin containing implant.

In yet another embodiment, the implant includes a material suitable for use in vivo within a subject in combination with at least two osteopontin active polypeptides,

wherein the active polypeptides are attached to the material such that upon implantation into the subject the osteopontin containing implant induces new bone formation.

In another embodiment, the implant includes a material suitable for use in vivo within a subject in combination with an osteopontin-derived peptide, wherein the osteopontin-derived peptide is attached to the material such that upon implantation into the subject the osteopontin-derived peptide containing implant induces new bone formation.

In another embodiment, the implant includes a material suitable for use in vivo within a subject in combination with a releasable form of osteopontin,

wherein the osteopontin is attached to the material such that upon implantation into the subject the osteopontin containing implant induces new bone formation.

In another aspect the invention features an osteopontin containing titanium implant. The implant includes a releasable form of phosphorylated osteopontin in combination with titanium suitable for use in vivo within a subject forming an osteopontin containing titanium implant.

In another aspect the invention features an osteopontin-derived peptide containing titanium implant. The implant includes an osteopontin-derived peptide in combination with titanium suitable for use in vivo within a subject forming an osteopontin-derived peptide containing titanium implant.

In yet another aspect the invention features a method of coating an implant with osteopontin, an active fragment thereof, or an osteopontin-derived peptide. The method includes non-covalently or electrostatically attaching osteopontin, an active fragment thereof, or an osteopontin-derived peptide to a surface of an implant, wherein the osteopontin, an active fragment thereof, or an osteopontin-derived peptide is attached to the surface of the implant such that it is releasable from the surface upon implantation into a subject.

The methods are useful in inducing new bone formation in a subject. The method includes implanting an implant, as described above, into a subject, wherein the osteopontin is released from the implant into the subject thereby inducing new bone formation in the subject.

In another embodiment an osteopontin containing cell recruitment system including a releasable osteopontin or a fragment thereof or a peptide derived therefrom in a form which provides a gradient and an implant used to form a cell recruitment system in the proximity of the implant, wherein the implant is targeted for cell recruitment by a gradient of osteopontin which forms in the proximity of the implant.

In another aspect the invention features a coated osseointegrator capable of implantation. The osseointegrator includes a coated material which is enhanced for osseointegration by at least about 100% when compared to an uncoated material based on the human osteoblast cell (HOS) attachment assay.

In another aspect the invention features a coated implant. The implant includes a coated material which increases the proliferation of osteoblasts by at least about 100% when compared to an uncoated material based on the human osteoblast cell (HOS) proliferation assay.

In still another aspect, the invention features a method for inducing new tissue formation in a subject at a site where tissue formation is needed. The method includes adding osteopontin or a fragment thereof or a peptide derived therefrom into a subject at a site where tissue formation is needed, wherein the osteopontin induces new tissue formation about the site.

In yet another aspect, the invention features an osteopontin glue which includes osteopontin, a mucopolysaccharide and a multivalent metal, e.g., calcium, magnesium or manganese. Preferably, the osteopontin is at a concentration of about 100 μg/g of glue.

In yet another embodiment, the invention features an isolated active osteopontin fragment or an osteopontin-derived fragment, for example, an active osteopontin fragment or an osteopontin-derived fragment having a cell attachment activity or active osteopontin fragment or an osteopontin-derived fragment having chemotactic activity. Preferred active osteopontin fragments and/or osteopontin-derived fragments include but are not limited to fragments or peptides including or having the sequence LVLDPK (SEQ ID NO: 2), or LVVDPK (SEQ ID NO: 3), petides or fragments including or having the sequence RGRDS (SEQ ID NO: 4), petides or fragments including or having the sequence X, X′, D, Z, Z1, wherein X and X′ are hydrophobic amino acids, D is aspartic acid, Z is proline (P), glycine (G), or serine (S), and Z′ is a basic amino acid, petides or fragments including or having the sequence GRGDS (SEQ ID NO: 5), petides or fragments including or having the sequence VFTPVVPTVDTYDGRGDSVVYGLRSKSKKFRR (SEQ ID NO: 6) or VFTPVVPTVDTYDGRGDSVVYGLRSKSKKFRRP (SEQ ID NO. 7), and petides or fragments including or having the sequence RSRRATEVFTPVVPTVDTYDGRGDSVVYGLRSKSKKFRRP (SEQ ID NO:8) (herein referred to as “OC-1016”).

In yet another embodiment, the invention features isolated peptide fragments having a cell attachment activity and/or cell spread activity. Preferred peptides include but are not limited to fragments or peptides including or having the sequence SDELVTDFPTDLPATEVFTPVVPTVDTYDGRGDSVVYGLRSKSKKFR RP (SEQ ID NO:9), RSRRATEVFTPVVPTVDTYDGRGDSVVYGRRSKSKKFRRP (SEQ ID NO:10), RSRRATEVFTPVVPTVDTYDGRGDSVVYGRRSKSKKFRRPAGAAGG PAGPAG PAGPAGPAGPA (SEQ ID NO:11), RSRRVFTPFIPTESANDGRGDSVAYGLKSKSKKFRR (SEQ ID NO:12), DTFTPIVPTVDVPNGRFDSLAYGLKSKSKKFQ (SEQ ID NO:13), RSRRATEVFTPVVPTVDTYDGRADSVVYGRRSKSKKFRRP (SEQ ID NO:14), or acetyl-RSRRATEVFTPVVPTVDTYDGRGDSVVYGLRSKSKKFRRP (SEQ ID NO:15) (herein referred to as “modified OC-1016” or “mOC-1016”).

The invention also pertains to peptides and osteopontin derived peptides and their regulatory activities pertaining to cellular spreading, chemotaxis, haptotaxis, and differentiation. Examples of cell types include, but are not limited to, osteoprogenitor cells, tumor cells, macrophages, periosteal cells, endothelial cells, epithelial cells, eosinophils and more generally, stem cells, limited potential precursor cells, precursor cells, committed precursor cells, and differentiated cells. The peptides, including OC-1016 and mOC-1016, are also active as anti-inflammatory agents.

The present invention is directed to isolated peptide molecules, their ability to bind to cells and modulate various cellular processes including differentiation. The present invention also pertains to antibodies, in particular monoclonal antibodies, which specifically bind to two forms of an osteopontin derived peptide (OC-1016 and mOC-1016, discussed below).

In another aspect, the invention features human osteoblast cell lines in which osteopontin expression is controlled by a constitutive promoter. One of skill will realize that given what is known in the art, osteopontin expression may also be expressed from a regulatable promoter. The regulation of such expression, or constitutive expression, will provide one with ability to modulate cellular processes including cell spreading, chemo- and hapto-taxis and therefore an indirect capability to influence wound healing, immune responses, bone development, tissue remodeling, and metastasis.

In another aspect of the invention, an osteopontin derived peptide is provided that binds to eosinophils. Eosinophil interacting peptides provide one with a mechanism to modulate eosinophil activity and therefore influence binding and recruitment behavior of eosinophils.

In another embodiment, the invention provides peptides that may be used in a method for promoting cell migration or cell differentiation to or in a target site, respectively. A therapeutically effective amount of the peptide(s) such that migration of a desired cell type to a target site is promoted. The peptides may be delivered or injected into the target site.

In yet another aspect of the invention, one may deliver material to a target site or region which is coated with the peptide that binds to cells and promotes their migration.

These examples demonstrate that OC-1016 and mOC-1016 enhance osseointegration in vivo, as well as induce cellular proliferation and spread.

### DETAILED DESCRIPTION OF THE DRAWINGS

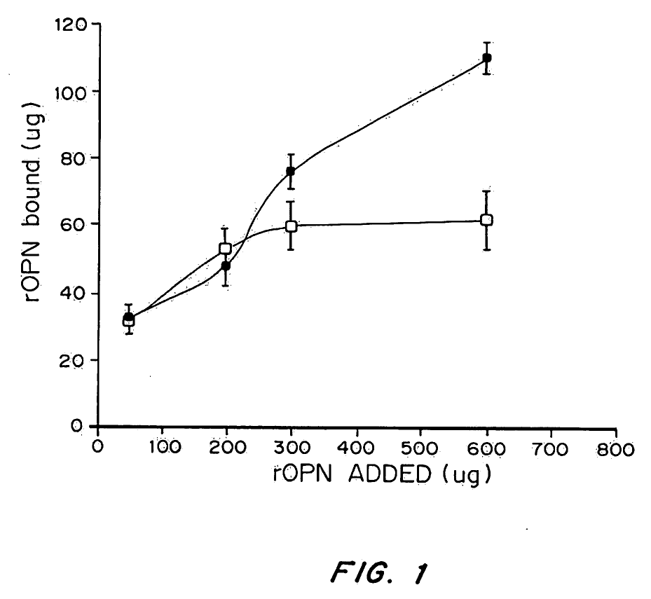

FIG. 1 a graph depicting the effect of Ca++ ions on the binding of osteopontin to Titanium disks.

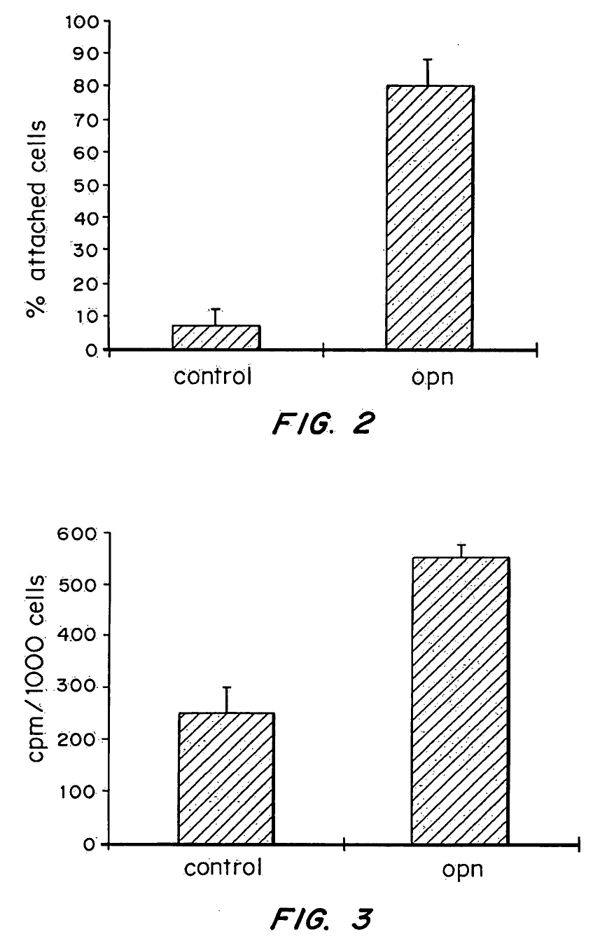

FIG. 2 is a bar graph depicting the effect of rhOPN on cell attachment to Titanium.

FIG. 3 is a bar graph depicting the effect of rhOPN bound to Titanium on cell proliferation.

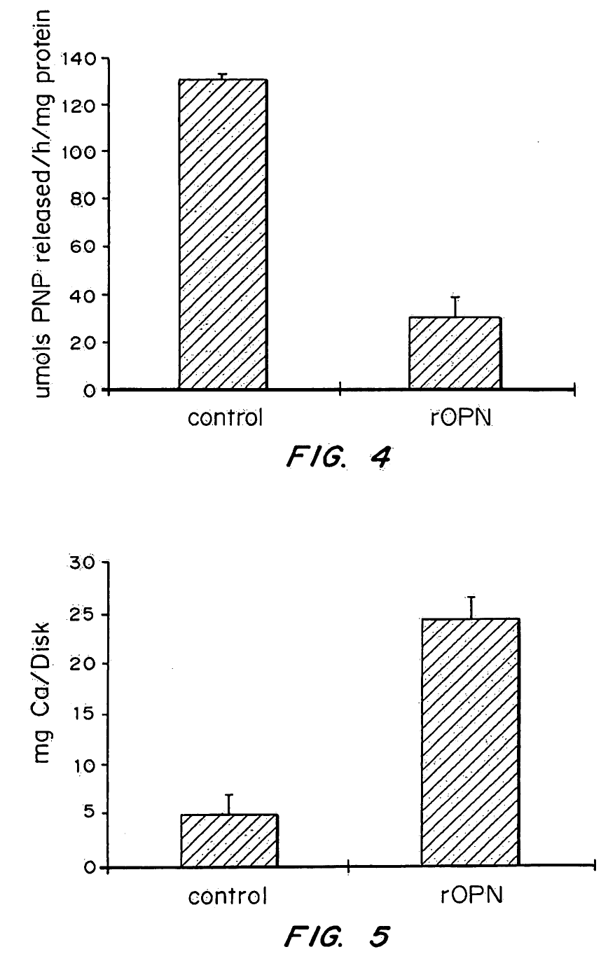

FIG. 4 is a bar graph depicting Apase activity of cells on coated and uncoated Titanium.

FIG. 5 is a bar graph depicting mineral content of human osteoblast cell culture.

### DETAILED DESCRIPTION OF THE INVENTION

An osteopontin coated implant includes a material suitable for use in vivo within a subject in combination with a releasable form of osteopontin forming an osteopontin containing implant.

As used herein, the term “material,” refers to a material suitable for use in vivo in a subject, e.g., a human or an animal subject, and capable of being part of an implant with osteopontin or a fragment thereof, e.g., releasable osteopontin. There are many art recognized materials suitable for use in vivo. These material include, but are not limited to, titanium, tantalum, Vitallium™, glass, plastic, chromocobalt (CrCo), stainless steel, natural or synthetic polymers such as collagen, cellulose, dextran or teflon beads.

As used herein, the term “osteopontin” or “osteopontin polypeptide,” refers to a form of osteopontin or a fragment thereof capable of performing its intended function in vivo, e.g., a form capable of influencing early bone matrix organization and mineralization through a cell, e.g., osteoblast or osteoclast, attachment. Examples of osteopontin forms include a phosphorylated osteopontin, e.g., an osteopontin having about 6 to about 12 phosphates per mol of protein, preferably, an osteopontin phosphorylated at one or more of the following amino acids selected from the group consisting of Ser26, Ser27, Ser63, Ser76, Ser78, Ser81, Ser99, Ser102, Ser105, Ser108, Ser117, and, preferably Thr138, and most preferably Thr152, a recombinant osteopontin, e.g., a human or murine recombinant osteopontin, e.g., the osteopontin secreted from murine B3H cells, and a naturally occurring osteopontin, e.g., the naturally occurring human osteopontin secreted from human osteoblast cells (SEQ ID NO:1). In a preferred embodiment threonine 152 is phosphorylated. In a more preferred embodiment, Ser26, Ser27, Ser81, Thr152 and Ser308 are phosphorylated.

As used herein, the term “active osteopontin peptide” refers to an osteopontin fragment that possesses at least one biological activity of a naturally occurring osteopontin. Preferred peptides include, but are not limited to, peptides having a chemotactic activity referred to herein as chemotactic peptides, e.g., peptides which comprise the amino acid sequence LVLDPK (SEQ ID NO: 2), or LVVDPK (SEQ ID NO: 3), or having a cell attachment activity referred to herein as cell attachment peptides, e.g., peptides which comprise the amino acid sequence RGRDS (SEQ ID NO: 4). In preferred embodiments, the osteopontin peptides can be coated onto the material via covalent, non-covalent, or electrostatic interactions.

In an exemplary embodiment, a chemotactic peptide can be a peptide which comprises an amino acid sequence X, X′, D, Z, Z1, wherein X and X′ are hydrophobic amino acids, D is aspartic acid, Z is proline (P), glycine (G), or serine (S), and Z′ is a basic amino acid.

Preferred hydrophobic amino acids include asparagine (N), leucine (L), valine (V), isoleucine (I), glutamine (Q), or methionine (M). Preferred basic amino acid residues include lysine (K) and arginine (R). In one embodiment X and X′ are selected from the group consisting of L, V, I, Q, M; Z is P, G, or S; and Z′ is either K or R. In a most preferred embodiment X is L, X′is L, Z is G, and Z′is K.

In another embodiment, a cell attachment peptide comprises the sequence GRGDS (SEQ ID NO: 5). GRGDS is a cell-binding domain which enhances cell attachment. In another embodiment, a cell attachment peptide has the sequence GRGDS (SEQ ID NO: 5). Preferred active peptides comprise the amino acid sequence VFTPVVPTVDTYDGRGDSVVYGLRSKSKKFRR (SEQ ID NO: 6); VFTPVVPTVDTYDGRGDSVVYGLRSKSKKFRRP (SEQ ID NO: 7); SDELVTDFPTDLPATEVFTPVVPTVDTYDGRGDSVVYGLRSKSKKFR RP (SEQ ID NO: 9); RSRRATEVFTPVVPTVDTYDGRGDSVVYGRRSKSKKFRRP (SEQ ID NO:10); RSRRATEVFTPVVPTVDTYDGRGDSVVYGRRSKSKKFRRPAGAAGG PAGPAG PAGPAGPAGPA (SEQ ID NO:11); RSRRVFTPFIPTESANDGRGDSVAYGLKSKSKKFRR (SEQ ID NO:12); and acetyl-RSRRATEVFTPVVPTVDTYDGRGDSVVYGLRSKSKKFRRP (SEQ ID NO:15) (m-OC-1016).

In another embodiment, a preferred active peptide does not have an “RGD” sequence. For example, a preferred active peptide has the amino acid sequence DTFTPIVPTVDVPNGRFDSLAYGLKSKSKKFQ (SEQ ID NO:13). Yet another preferred active peptide has the amino acid sequence RSRRATEVFTPVVPTVDTYDGRADSVVYGRRSKSKKFRRP (SEQ ID NO:14).

As used herein, an osteopontin-derived peptide includes an “active osteopontin peptide” as defined herein, including about one, two, three, four, five, six, seven, eight, nine or ten residues which differ from the amino acid residues present in a naturally occurring active osteopontin peptide, the residues not interfering with the activity of the active peptide. For example, residues can be added at the C- or N-terminus of an active osteopontin peptide (e.g., to facilitate purification of the active peptide). Alternatively, a relatively few number of residues can be substituted within the consecutive sequence of the active peptide (e.g., substituted within the sequence of a naturally-occurring osteopontin sequence), the substitutions not interfering with the activity of the active peptide. A preferred osteopontin-derived peptide comprises the amino acid sequence RSRRATEVFTPVVPTVDTYDGRGDSVVYGLRSKSKKFRRP (SEQ ID NO: 8). Alternatively, any type of chemical modification may be incorporated into or attached to any of the peptides. The chemical modification of choice will not interfere with the activity of the peptide. SEQ ID NO:15 (mOC-1016) represents one such example.

As used herein, the phrase “in a releasable form,” is intended to include osteopontin coated on top of the material in such a way that an osteopontin or a fragment thereof or a peptide derived therefrom is capable of being released from the surface of the implant and performing its intended function in vivo, e.g., it is capable of establishing an osteopontin gradient in the proximity of an implant, preferably, within about 24 hours, more preferably within about 48 hours, of implantation. As used herein, “osteopontin gradient,” refers to a protein gradient which results in the recruitment of cells, e.g., osteoblasts or osteoclasts, to an implant. Preferably, the osteopontin is non-covalently or electrostatically attached to the material. Non-covalent attachment is known in the art and includes, but is not limited to, attachment via a divalent ion bridge, e.g., a Ca++, Mg++ or Mn++ bridge; attachment via absorption of osteopontin or a fragment thereof or a peptide derived therefrom to the material; attachment via plasma spraying or coat drying of a polyamine, e.g., polylysine, polyarginine, spermine, spermidine or cadaverin, onto the material; attachment via a second polypeptide, e.g., fibronectin or collagen, coated onto the material; or attachment via a bifunctional crosslinker, e.g., N-Hydroxysulfosuccinimidyl-4-azidosalicylic acid (Sulfo-NHS-ASA), Sulfosuccinimidyl(4-azidosalicylamido)hexanoate (Sulfo-NHS-LC-ASA), N-γ-maleimidobutyryloxysuccinimide ester (GMBS), N-γ-maleimidobutyryloxysulfosuccinimide ester (Sulfo-GMBS), 4-Succinimidyloxycarbonyl-α-methyl-α-(2-pyridyldithio)-toluene (SMPT), Sulfosuccinimidyl 6[α-methyl-α(2-pyridyldithio)toluamido]hexanoate (Sulfo-LC-SMPT), N-Succinimidyl-3-(2-pyridyldithio)propionate (SPDP), Succinimidyl 6[3-(2-pyridyldithio)propionamido]hexanoate (LC-SPDP), Sulfosuccinimidyl 6-[3-(2-pyridyldithio)propionamido]hexanoate (Sulfo-LC-SPDP), Succinimidyl 4-(N-maleimidomethyl)cyclohexane-1-carboxylate (SMCC), Sulfosuccinimidyl 4-(N-maleimidomethyl)cyclohexane-1-carboxylate (Sulfo-SMCC), m-Maleimidobenzoyl-N-hydroxysuccinimide ester (MBS), m-Maleimidobenzoyl-N-hydroxysulfosuccinimide ester (Sulfo MBS), N-Succinimidy(4-iodoacetyl)amino benzoate (SIAB), Sulfosuccinimidyl(4-iodoacetyl)amino benzoate (Sulfo-SIAB), Succinimidyl 4-(ρ-maleimidophenyl) butyrate (SMPB), Sulfosuccinimidyl 4(ρ-maleimidophenyl) butyrate (Sulfo-SMPB), or Azidobenzoyl hydrazide (ABH), to the material. In other preferred embodiments osteopontin or a fragment thereof or a peptide derived therefrom is attached to the material via an electrostatic interaction.

Alternatively, the osteopontin can be attached to an implant for tissue surface via non-covalent attachment, as described above, further including a mucopolysaccharide. Mucopolysaccharides are art recognized and include glycosaminoglycans having, for example, repeating units of N-acetylchondrosine or β 1-3 glucuronidic and β 1-4 gluconsaminidic groups. Suitable mucopolysaccharides include chondroitin sulfate or hyaluronic acid. Preferably, hyaluronic acid is greater than a disaccharide; the hyaluronic acid has a molecular weight range of less than 100 kDa, more preferably between about 20 to about 100 kDa, e.g. between about 50-100, 70-100, or 30-80 kDa.

As used herein, the term “implant,” refers to a surgical implant suitable for use in vivo and where it would be desirable to have osteopontin for promoting cell, e.g., osteoblast or osteoclast, attachment. Examples of suitable implants include but are not limited to dental implants, e.g., dental screws or fixtures, jaw modification implants, face reconstruction implants, orthopedic implants, e.g., orthopedic screws, rods or joints, e.g., hip or knee replacement implants. A preferred implant is a titanium dental implant.

As used herein, the phrase “an osteopontin containing cell recruitment system” refers to a system in which osteopontin or a fragment thereof or a peptide derived therefrom is introduced into a subject independent of an implant. Preferably, the osteopontin or a fragment thereof or a peptide derived therefrom is introduced in the proximity of an implant in a form of a gel or a sponge. In other preferred embodiments, the osteopontin or a fragment thereof or a peptide derived therefrom contained in a gel or a sponge is capable of generating a gradient of osteopontin in the proximity of an implant such that cells, e.g., osteoblasts or osteoclasts, are recruited to the implant. The phrase “an osteopontin containing cell recruitment system” is also intended to include chemotactic effects of osteopontin in facilitating wound healing and stimulating the recruitment of tissue remodeling cells from surrounding tissues. Tissue remodeling cells include mesenchymal, macrophage and granulocytes. Wound healing cells include, for example, cytokines which include TGFB and growth factors, cell-stimulating molecules and healing cells such as macrophages which help to clear chronic necrotic tissue from damaged tissue area.

The term “mesenchymal cell” is art recognized and is intended to include undifferentiated cells found in mesenchymal tissue, e.g., undifferentiated tissue composed of branching cells embedded in a fluid matrix which is responsible for the production of connective tissue, blood vessels, blood, lymphatic system and differentiates into various specialized connective tissues.

The term “growth factors” is art recognized and is intended to include, but is not limited to, one or more of platelet derived growth factors (PDGF), e.g., PDGF AA, PDGF BB; insulin-like growth factors (IGF), e.g., IGF-I, IGF-II; fibroblast growth factors (FGF), e.g., acidic FGF, basic FGF, β-endothelial cell growth factor, FGF 4, FGF 5, FGF 6, FGF 7, FGF 8, and FGF 9; transforming growth factors (TGF), e.g., TGF-β1, TGF-β1.2, TGF-β 2, TGF-β3, TGF-β5; bone morphogenic proteins (BMP), e.g., BMP 1, BMP 2, BMP 3, BMP 4; vascular endothelial growth factors (VEGF), e.g., VEGF, placenta growth factor; epidermal growth factors (EGF), e.g., EGF, amphiregulin, betacellulin, heparin binding EGF; interleukins, e.g., IL-1, IL-2, IL-3, IL-4, IL-5, IL-6, IL-7, IL-8, IL-9, IL-10, IL-11, IL-12, IL-13, IL-14; colony stimulating factors (CSF), e.g., CSF-G, CSF-GM, CSF-M; nerve growth factor (NGF); stem cell factor; hepatocyte growth factor, and ciliary neurotrophic factor. Adams et al., “Regulation of Development and Differentiation by the Extracellular Matrix” Development Vol. 117, p. 1183-1198 (1993) (hereinafter “Adams et al.”) and Kreis et al. editors of the book entitled “Guidebook to the Extracellular Matrix and Adhesion Proteins,” Oxford University Press (1993) (hereinafter “Kreis et al.”) describe extracellular matrix components that regulate differentiation and development. Further, Adams et al. disclose examples of association of growth factors with extracellular matrix proteins and that the extracellular matrix is an important part of the micro-environment and, in collaboration with growth factors, plays a central role in regulating differentiation and development.

As used herein, the phrase “inducing new bone formation,” refers to a process which results in attachment, proliferation and/or differentiation of bone cells, e.g., osteoblasts and/or osteoclasts, and subsequent bone mineralization, in the proximity of an implant.

As used herein, the phrase “a coated osseointegrator capable of implantation,” refers to a coated material which when implanted into a subject in vivo enhances osseointegration in the vicinity of the coated material by at least about 100% when compared to an uncoated material. Preferably, the coated material is a material coated with an osteopontin or a fragment thereof or a peptide derived therefrom, as described herein. In other preferred embodiments, the rate of osseointegration is enhanced by at least about 300%, 500%, 800%, 1000%, 1100% or 1200%, when compared to an uncoated material. The percentage values intermediate to those listed also are intended to be part of this invention, e.g., 350%, 875%, or 1150%. Rate of osseointegration can be measured using the human osteoblast cell (HOS) attachment assay as described in Examples 2 and 7 below, or by other methods known to those of skill in the art.

As used herein, the term “coated implant,” refers to a coated material which when implanted into a subject in vivo increases the proliferation of osteoblasts in the vicinity of the coated material by at least about 100% when compared to an uncoated material. Preferably, the coated material is a material coated with an osteopontin or a fragment thereof or a peptide derived therefrom, as described herein. In other preferred embodiments, the rate of proliferation is increased by at least about 50%, more preferably by at least about 200%, when compared to an uncoated material. The percentage values intermediate to those listed also are intended to be part of this invention, e.g., 75%, 125% or 150%. Rate of proliferation can be measured using the human osteoblast cell (HOS) proliferation assay as described in Examples 3 and 8 below, or by other methods known to those of skill in the art.

The present invention is also directed to methods for inducing new tissue formation in a subject at a site where tissue formation is required. The methods include adding osteopontin into a subject at a site where tissue formation is needed, wherein the osteopontin induces new tissue formation about the site. In a preferred embodiment the osteopontin is a recombinant osteopontin. In a most preferred embodiment, the site includes an implant as described herein.

A osteopontin glue includes osteopontin, a mucopolysaccharide and a multivalent metal. Suitable multivalent metals include copper, zinc, barium, calcium, magnesium, and manganese. The osteopontin glue can be administered to an area of tissue in need of repair, e.g., a wound, a cut, or other damaged tissue area, e.g., necrotic tissue. The osteopontin glue can be administered by methods known to those skilled in the art, such as, via injection. Administration of the osteopontin glue enhances tissue regeneration with concomitant removal of necrotic cells. In a preferred embodiment, the osteopontin glue can be used with an implant as described herein.

They are administered in tablets or capsule form, by injection, inhalation, eye lotion, ointment, suppository, etc. administration by injection, infusion or inhalation; topical by lotion or ointment; and rectal by suppositories. Injection or topical application is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Actual dosage levels of the active ingredients in the pharmaceutical compositions may be varied so as to obtain an amount of the active ingredients which are effective to achieve the desired therapeutic response for a particular patient, composition, and mode of administration, without being toxic to the patient.

The selected dosage level will depend upon a variety of factors including the activity of osteopontin of the present invention employed, the route of administration, the time of administration, the rate of excretion of the osteopontin being employed, the duration of the treatment, other drugs, compounds and/or materials used in combination with the osteopontin employed, the age, sex, weight, condition, general health and prior medical history of the patient being treated, and like factors well known in the medical arts. In a preferred embodiment the concentration of osteopontin in the glue is between about 0.1 μg to about 100 μg, preferably about 100 μg/g of carrier.

Not wishing to be bound by theory, it is believed that the osteopontin glue provides a mechanism for “laminating” tissue to tissue or tissue to implant. A plausible explanation for glue's ability to facilitate tissue reconstruction or repair is as follows: Mucopolysaccharides include both hydrophobic and hydrophilic domains, for example, which can coat, e.g., adhere to, the surface of implant or tissue. The mucopolysaccharide provides ionic charge for a multivalent cation to interact with the mucopolysaccharide, acting as a bridge between the implant surface and osteopontin. Once the osteopontin is within the region where cell-recruitment is required, the osteopontin helps to facilitate the regeneration of the tissue in the gradient area of the osteopontin. Alternatively, an implant surface may be oxidized so that the multivalent metal can bind with the oxidized surface, thus providing a bridge directly to the osteopontin. It can be envisioned that interactions between the osteopontin and further layers of mucopolysaccharides can further produce a laminating effect for multiple layers of mucopolysaccharide, multivalent metal, osteopontin.

The phrase “pharmaceutically acceptable carrier” is art recognized and includes a pharmaceutically acceptable material, composition or carrier, suitable for administering osteopontin compositions of the invention to mammals by injection. The vehicles include liquid or solid filler, diluent, excipient, solvent or encapsulating material, involved in carrying or transporting the bone precursor composition from a syringe to the cavity in need thereof. Each carrier must be “acceptable” in the sense of being compatible with the other ingredients of the formulation and not injurious to the patient. Some examples of materials which can serve as pharmaceutically acceptable vehicles, include: sugars, such as lactose, glucose and sucrose; starches such as cornstarch and potato starch; cellulose and its derivatives, such as sodium carboxy methylcellulose, ethyl cellulose and cellulose acetate; powdered tragacanth; malt; gelatin; talc; excipients, such as cocoa butter and suppository waxes; oils such as peanut oil, cottonseed oil, safflower oil, sesame oil, olive oil, corn oil and soybean oil; glycol such as propylene glycol; polyols such as glycerin, sorbitol, manitol and polyethylene glycol; esters, such as ethyl oleate and ethyl laurate; buffering agents such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants such as sodium lauryl sulfate and magnesium stearate, as well as coloring agents, stabilizers, preservatives or antioxidants can also be present in the compositions.

Methods of preparing these formulations or compositions include the step of bringing into association the osteopontin glue compositions of the present invention with a carrier and, optionally, one or more accessory ingredients. In general, the formulations are prepared by uniformly and intimately bringing into association the components of the osteopontin glue of the present invention with the carrier.

Liquid dosage forms suitable for administration of the osteopontin glue compositions of the invention include pharmaceutically acceptable emulsions and microemulsions, solutions, suspensions, syrups and elixirs. In addition to the active ingredients, e.g. osteopontin, multivalent metals and mucopolysaccharides, the liquid dosage form can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propyleneglycol, 1,3-butyleneglycol, oils (in particular, cottonseed, groundnut, corn, germ, olive, castor and sesame oils), glycerol, tetrahydrofuryl alcohol, polyethyleneglycols and fatty acid esters, sorbitan and mixtures thereof.

The osteopontin compositions can also contain adjutants such as preservatives, wetting agents, emulsifying agents and dispersing agents. Prevention of the action of microorganisms may be insured by the inclusion of various anti-bacterial and anti-fungal agents, for example, paraben, chlorobutanol, and phenol sorbic acid. It may also be desirable to include isotonic agents, sugars, or salts such as sodium chloride. In addition, prolonged absorption of the osteopontin compositions can be brought about by the inclusion of agents which allay absorption such as aluminum monosterate and gelatin e.g., collagen.

In Vitro Modification of Osteopontin

Phosphorylation of Osteopontin:

Both natural and recombinant osteopontin can be modified by phosphorylation of the amino acid sequence encoding native osteopontin. The osteopontin can be modified so that phosphorylation is present in the absence of, or with altered glycosylation. The osteopontin can also be modified so that it has less phosphorylation or more phosphorylation than native forms of osteopontin, or is phosphorylated at sites other than those which are naturally phosphorylated.

Phosphorylation is achieved by incubation of the osteopontin in the presence of either eucaryotic kinases such as casein kinase type II or cAMP-dependent kinases. These kinases can be obtained from cytosolic or microsomal extracts, or in purified or semi-purified form from sources such as Sigma Chemical Co., Inc., or as described in the literature. As described in the example below, at least three different kinase preparations from mouse kidney could be used to phosphorylated osteopontin in vitro. These preparations contain a mixture of kinase activities, several of which can phosphorylate the fusion protein. Casein kinase I, casein kinase II and mammary gland casein kinase participate in hierarchical phosphorylation reactions. Phosphorylation of one site by any of these kinases may affect phosphorylation at another site by a different kinase.

As further demonstrated by the examples below, osteopontin appears to be a complex substrate with at least 58 consensus phosphorylation sites for different types of kinases, as shown in Table I. These putative phosphorylation sites are not randomly distributed throughout the protein but appear as if they were organized in eight clusters. For example, between residues 100 and 126 there are 9 potential phosphorylation sites for either casein kinase I, casein kinase II or mammary gland casein kinase. In addition to potential phosphorylation sites for these independent casein kinase family of enzymes, osteopontin also contains potential phosphorylation sites for cAMP- and cGMP-dependent protein kinases, calmodulin-dependent protein kinase, and protein kinase c. There are several fold more potential phosphorylation sites in recombinant osteopontin than those found phosphorylated in osteopontin isolated from bone. Not all of the potential sites may be phosphorylated at any given time, since some sites may be not accessible to protein kinases or some tissues may not contain all of the kinase activities required for the phosphorylation of osteopontin. Furthermore, the clustering of sites suggests that certain phosphorylated residues can serve as specificity determinants. For example, phosphorylation of a Ser/Thr residue by any kinase can generate a site for phosphorylation of an adjacent phosphorytable residue by either casein kinase I or mammary gland casein kinase. Conversely, phosphorylation at one site by a particular kinase may suppress the phosphorylation of a nearby residue, such as the mutually exclusive phosphorylation of hormone-sensitive lipase by cAMP-dependent protein kinase and calmodulin-dependent protein kinase.

Further modifications on the site and extent of phosphorylation can be achieved by expression of osteopontins with altered structures by differential splicing and post-translational modifications, as well as by the use of fragments and site-specific mutations at any one of these phosphorylation sites.

For phosphorylation by calcium/calmodulin kinase II, the reactions are carried out in the presence of 1.5 mM CaCl₂ and 3 μg calmodulin. For phosphorylation by protein kinase C, the reactions are carried out in the presence of 8 μg/ml phosphatidylserine, 0.8 μg/ml of diacylglycerol, and 1 mM CaCl₂. For autophosphorylation the reaction is carried out in the presence of 10 mM MnCl₂. For phosphorylation by cGMP dependent protein kinase the reactions are carried out in the presence of 0.1 μM cGMP. No additions are necessary for the phosphorylation of osteopontin by casein kinase I or mammary gland casein kinase.

Determination of Phosphorylation Sites in Osteopontin:

After phosphorylation with ³²P-ATP and the desired kinase, osteopontin is digested with either trypsin, endopeptidase Glu-C, or endopeptidase Asp-N. The resulting peptides are separated by HPLC and the radiolabeled peptides sequenced. The position of the phosphorylated residue is determined by the coelution of radioactivity with the amino acid in that cycle.

Dephosphorylation of Osteopontin:

Osteopontin can be dephosphorylated by incubating the protein in either 100 μl 20 mM HEPES buffer, pH 8.5, and 1 unit of alkaline phosphatase, or 100 μl 20 mM acetate buffer pH, 5.0 and 1 unit of acid phosphatase, for several hours. Osteopontin can also be dephosphorylated by incubating the phosphoprotein with between 0.1 and 1 units of protein phosphatase 2A at 4° C. for 1 h. Osteopontin can be also dephosphorylated by incubating the protein in 0.1 N NaOH for 1 h at 37° C.

TABLE 1
Predicted phosphorylation sites in Osteopontin

| | Position of |
|---|---|
| Protein | phosphorylated |
| Kinase | residue |
| Casein Kinase I | 239, 275, 280, 308 |
| | 26, 76, 78, 99, 102, 105, 108, |
| | 117, 120, 123, 126, 129, 234, 308 |
| Casein Kinase II | 26, 27, 62, 63, 191, 215, 228, 280, 291 |
| | 76, 237 |
| Ca/Calmodulin- | 162, 171 |
| dependent | |
| Protein Kinase II | |
| cGMP-Dependent | 24, 73, 81, 162, 169, 171, 243, 270, 275, 303 |
| Protein Kinase | |
| cAMP-Dependent | 224, 243, 270 |
| Protein Kinase | |
| Protein Kinase C | 49, 239, 171 |
| Tyrosine Kinase | 165 |
| Proline-Dependent | 147 |
| Protein Kinase | |

Glycosylation:

N-glycosylation of Osteopontin:

Osteopontin can be N-glycosylated using colichol-P—P-oligosaccharide and microsomal oligosaccaride transferase. The oligosaccharide side chain can be further processed by using enriched golgi preparations and the appropriate UDP-saccharides.

O-glycosylation of Osteopontin:

Osteopontin will be O-glycosylated by incubating the protein with commercially available rabbit reticulocyte lysate, which has been demonstrated by glycosylate nascent proteins in vitro (e.g., Starr, S. M. and Hanover, J. A. (1990) J. Biol. Chem. 265:6868-6873). Alternatively osteopontin could be O-glycosylated by using purified UDP-GalNAc:polypeptide N-acetylglactosaminyltransferase and UDP-N-acetylgalactosamine. The resulting O-glycosylated protein could be used to build more complex oligosaccharide side chains, using purified transferases and the appropriate sugar derivatives.

Glycation of Osteopontin (Nonenzymatic):

Non-enzymatic glycation involves the condensation of any sugar aldehyde or ketone, including phosphorylated derivatives of sugars, with either an α or ε amino group, resulting first in the rapid formation of a Schiff base. The Schiff base adduct can subsequently rearrange to the more stable Amadoriri product. For example, incubation of osteopontin with glucose, for several hours, will result in the formation β-pyranosyl Schiff base adduct, which will rearrange, with time, to the β-furanosyl Amadori product. Alternatively, the α-pyranosyl Schiff base adduct can be reduced at for 1 h at 22° C. with 0.1% sodium horohydride to yield 1-deoxy-1-aminosorbitol derivative.

Sialation of Osteopontin:

O-glycosylated osteopontin can be modified further by the addition of sialic acid. Briefly, 200 μg of osteopontin will be incubated with 0.5 milliunits of α 2,3-sialyltransferase in 100 μl 20 mM HEPES buffer pH, 6.5, containing varying concentrations of CMP-sialic acid for 1 h at 37° C. N-glycosylated osteopontin can be sialated using α 2,6-sialytransferase and the conditions described above.

Deglycosylation of Naturally Occurring Osteopontin:

Osteopontin, isolated from tissues, can be deglycosylated by the following methods:

Removal of N-linked Oligosaccharides:

After treatment of osteopontin with neuranimidase to remove sialic acids, osteopontin is incubated overnight with 0.3 units of N-glycanase (Genzyme, Boston, Mass.) 100 μl of 20 mM HEPES buffer, pH 7.5, at 37° C.

Removal of O-linked Oligosaccharides:

Asialoosteopontin is incubated for 1 to 6 h with 4 milliunits o-glycanase (Genzyme, Boston, Mass.) in 100 μl of 20 mM MOPS buffer, pH 6.0, at 37° C.

Removal of Oligosaccharides from Osteopontin:

Total deglycosylation of osteopontin can be achieved by incubating the protein with 0.1% anhydrous trifluoromethanesulphonic acid (TFMS) for several hours. This treatment removes both O- and N-linked oligosaccharides.

Sulfation of Osteopontin:

Sulfation of osteopontin and its derivatives is accomplished using the procedure described by Varahabahotla, et al. (1988) BBA, 966:287-296, using the enzyme sulfotransferase and 3′-phosphoadenosine-5′-phosphosulfate as the sulfate donor. Osteopontin contains 4 tyrosines. The sulfated proteins are then purified by gel permeation chromatography.

Titanium

1 Titanium Surface Characteristics

Titanium (Ti) reacts immediately with oxygen when exposed to air. In less than a millisecond an oxide layer greater than 10A is formed, and within a minute the oxide thickness will be of the order of 50 to 100A (Kasemo B, J. Of Prosth Dent. 49(6):832-837, 1983). Ultrasonic cleaning and autocleaving involves additional growth of the surface oxide, as well as probable incorporation of OH radicals in the oxide (Kasemo B, J. Of Prosth Dent. 49(6):832-837, 1983). Titanium forms several stable oxides such as TiO₂, TiO, and Ti₂O₃, with TiO₂ being the most common one. All oxides have high dielectric constants (higher than for most other metal oxides) in the range of 50 to 120. For these reasons a single stoichiometric oxide is not expected to form on the implant surface. The oxide might be called TiOx, where x gives the average oxygen content of the oxide. The tissue implant reaction is thus a reaction with TiO₂ at the implant surface and not with the element titanium as such (Kasemo B, J. Of Prosth Dent. 49(6):832-837, 1983).

Titanium dioxide has physical/chemical characteristics that differ from metallic titanium; characteristics which are more closely related to ceramics than to metals (LeGeros R Z and Craig R G, J. Of Bone and Mineral Research 8(2):s583-s593, 1993). TiO is bioinert, Ti is biotolerant (LeGeros R Z and Craig R G, J. Of Bone and Mineral Research 8(2):s583-s593, 1993). Biomaterial composition affects surface chemistry and tissue response. Bioinert materials, which include ceramic oxides (alumina, zirconia) and biotolerant materials (metal alloys and polymers) do not become directly attached to the bone, and consequently, the material bone interface is weaker in tension and shear strengths but not necessarily in compression loading.

It has been established that titanium oxide surfaces bind cations, particularly polyvalent cations (Abe M., Oxides and hydrous oxides of multivalent metals as inorganic ion exchangers, Inorganic Ion Exchange Materials (ed. A. Clearfield) CRC Press, Boca Raton, Fla., USA, pp 161-273, 1982). Titanium surfaces have a net negative charge at the pH values encountered in animal tissues, the pK being 4.0. This binding of cations is based on electrostatic interactions between titanium-linked 0- on the implant surface, and cations. The oxide layer is highly polar and attracts water and water-soluble molecules in general (Parsegian V A, J. Of Prosth Dent. 49(6):838-841, 1983).

2 The Bone-Titanium Layer

It is known that osseointegrated implants are characterized by the presence of an organic interfacial layer, containing no collagen fibrils, between the bone and the implant. This intervening layer in osseointegrated implants has been reported to stain with lanthanum and alcin blue and is both hyaluronidase and chondroitinase sensitive, suggesting proteoglycan content (Albrektsson T et al, Annals of Biomedical Engineering, 11, 1-27, 1983). The thickness of the glycan layer was found to vary with the biocompatibility of the implant material from 20 to 40 nm for Ti and 30 to 50 nm for zirconia (Albrektsson T, Jacobson M, J. Prosthet Dent 57:597-607, 1987). Establishment of this layer is reported to be critical for the success of the implant since it may provide an optimal interface between the dental implant and the newly formed bone (Nanci A et al, Cells and Materials, 4(1):1-30,1994).

Tissue response to commercially pure Titanium (cp Ti) was examined to characterize the bone implant interface. Lectin cytochemistry was used to detect glycoconjugates and immunocytochemistry for noncollagenous bone and plasma proteins. The composition of the titanium-matrix interface with that of natural bone interfaces such as cement lines and laminae limitantes was compared. The concentration of osteopontin (Opn) and alpha HS-glycoprotein at the bone titanium interface was consistent with the composition of cement lines at matrix-matrix interface and laminae limitantes at various cell-matrix interfaces. Furthermore, the data indicated that the interfacial layer between the bone and the implant is also rich in glycoconjugates containing sacharides such as galactose, a sugar residue found in relatively large proportion in osteopontin.

3 Bone Healing Around Ti

The idea of osseointegration arose from studies of bone wound healing. Titanium chambers containing a transillumination system were inserted into the fibulae of rabbits to observe cellular changes during endosteal wound healing. At the completion of the study, retrieval of the titanium chambers required fracture of bone tissue that was integrated into the chamber surface. This incidental finding became the basis for the use of Titanium in endosseuos implant construction (Branemark P-I, Introduction to osseointegration. In Branemark P-I, Zarb Ga, Aiberktsson T (eds) Tissue-Integrated prosthesis. Quintessence Publishing Co, Inc., Chicago, pp 11-76, 1985).

The bone trauma generated by implant placement is followed by clot formation, acute inflammation, recruitment and proliferation of stromal cells and their differentiation into osteogenic lineage cell, followed by filling the defect with and bone and finally mineralization of the matrix (O'Neal R B et al., J. Oral Implantol. 18:243-255, 1992). Throughout this process; macromolecules, including cytokines and adhesion molecules, that orchestrate the course of wound healing and osteogenesis, are secreted into the extracellular milieu (O'Neal et al, Biological requirements for material integration(1992). J. Oral Implantol. 18:243-255, 1992). The interaction of some of these macromolecules with the implant surface determines to a measurable extent how well the implant is integrated.

Early postoperative motion which can occur with an unstable device impairs bone regeneration leading instead for fibrous repair, encapsulation and chronic inflammation, which can further contribute to instability and more excessive motion. If the interface is not integrated, large shear displacements occurring across the interface may result in combined corrosion and wear (Galante J O et al., J. Of Orthopaedic Research 9:760-775, 1991).

The nature of the implant bone interface is also affected by the surface chemistry and topography of the implant. Since titanium does not induce bone formation, one way of assuring apposition of bone cells to the implant is to design an implant surface that is attractant to these molecules and/or supports osteomorphogenesis.

4 Changes On Macroscopic Characteristics Of Titanium

Steps to maximize integration have addressed the implant: Studies about surface of the implant clearly show that bone cells adhere securely onto Titanium surfaces, and rough-textured (acid) and porous-coated Ti surfaces enhance both the synthesis and mineralization of the extracellular matrix (Bowers K T et al., Int. J. of Oral and Max. Imp. 7(3):302-310, 1992, Groessner-Schreiber B, Tuan R S, J. Of Cell Science 101,209-217, 1992). Electrochemical potentials for porous conditions are relatively similar to those for smooth-surfaced conditions. However, corrosion rates are increased for porous conditions due to the added area per unit volume (Galante J O et al, J. Of Orthopaedic Research. 9:760-775, 1991).

Healing Of Bone Using Titanium Coated With Proteins Recent studies have focused on improving the osseointegration of implants into bone by coating the Ti surfaces of implants with various substances including hydroxyapatite (Klein C P et al., Biomaterials. 15(2): 146-50, 1994; Jansen J A et al., J. Biomedical Materials Res. 25(8):973-89, 1991; Holmes R E, Plast. Reconstr Surg 63:626-636, 1979), fibronectin (Rutherford R B et al., Int. J. Oral and Maxillofacial implants. 7(3):297-301,1992), and bone morphological proteins (BMP's) (Xiang W et al, Journal of Oral and Maxillofacial Surgery. 51(6):647-511, 993). Histological examinations of bone/titanium interface from such studies revealed various degrees of success in improving the osseointegration of Ti implants.

Titanium and Osteopontin

1 Protein Expression During Bone Formation

Morphological and histological studies on bone development categorize a linear sequence of cell differentiation progressing from an osteoprogenitor cell to preosteoblasts, osteoblasts and finally osteocytes and lining cells (Aubin J E et al., Analysis of osteoblast lineage and regulation of differentiation. In “Chemistry and Biology of Mineralized Tissue” (H. Slavkin and P Price, eds), pp 267-276. Excepta Medica, Amsterdam, 1992). Recently, the morphological and histological studies have been supplemented with the elucidation of some of the specific proteins secreted by bone cells at specific stages during their development. For example collagen type I is secreted by early and mature osteoblasts but decreases with late osteoblasts and osteocytes. Alkaline phosphatase is expressed by preosteoblasts and is accepted as a marker for osteoblasts. Osteopontin and bone sialoprotein are secreted by early osteoblasts, just prior to the onset of mineralization, but decreases as mineralization proceeds and osteoblasts mature and differentiate into osteocytes. Osteoblastic cells in vitro show an initial peak of Opn mRNA expression at early cultured times, followed by a second mayor peak of expression when the cultures begin to mineralize (Owen T A, J. Cell. Physiol. 143, 420-430, 1990; Strauss G P et al., J. Cell. Biol. 110,1368-1378, 1990). Osteocalcin is secreted by mature osteoblasts after the onset of mineralization. The order of appearance of proteins at bone interfaces, particularly with respect to type I collagen, is important in understanding the events leading to bone formation and turn over, and ultimately osseointegration.

2 Possible Role Of Osteopontin In Bone Formation

Osteopontin is a cell adhesion protein first identified in bone, but now associated with other tissues as well. Osteopontin is a phosphorylated glycoprotein containing an RGD cell-binding sequence. In mineralized tissues, OPN is expressed prior to mineralization and regulated by osteotropic hormones, binds to hydroxyapatite, and enhances osteoclast and osteoblast adhesion. Although the exact function of Opn is yet unknown, possibilities include a role in the recruitment of bone precursor cells to a site of mineralization, and a role in protection against bacterial infection (Butler W T, Connect. Tissue Res. 23,123-136, 1989).

Osteopontin in laminae limitantes at bone surfaces may act as a substrate for osteoclast adhesion, and then for initial sealing zone attachment, during osteoclast migration and bone matrix resorbtion, respectively. During the reversal phase of the remodeling sequencing, the initial expression of osteopontin has been suggested to reflect the involvement of this non-collagenous bone protein in cell-matrix interaction (Lian J B, Stein G S, Crit. Rev. Oral Biol. Med. 3, 269-305,1992). Opn secreted early in the life cycle of differentiating preosteoblasts accumulates at the resorbed bone surfaces to form a cement line. The deposition of this planar arrangement of Opn initially may serve to influence early matrix organization and mineralization, and possibly preosteoblasts adhesion at these sites. It also may function in a broader sense as a matrix-matrix/mineral biological glue to attach newly formed bone to older bone in order to maintain overall tissue integrity and biomechanical strength during bone remodeling (McKee M D, Nanci A, Osteopontin and the bone Remodeling Sequence Colloidal-Gold Immunocytichemistry of an Interfacial Extracellular Matrix Protein, In: Osteopontin:Role in Cell Signaling and Adhesion. Annals of the New York Academy Sciences 760: Apr. 21, 1995). Based on the sequence of appearance of matrix proteins, it may be postulated that Opn place a dual role, first participating in cells attachment and then in the mineralization of the cement line-like material found in vivo (Shen X, Cells and Materials 3, 257-272, 1993).

3 Bonding of Proteins to Titanium Surfaces

An implanted material attains and maintains contact with interfacial tissue through its surface. When a substrate or an implant is inserted into the body environment, it is exposed to cells and a host of ionic and molecular species that ultimately determine the course of interfacial events (Kasemo B, J. Of Prosth Dent. 49(6):832-837, 1983). One of the first things to happen is the absorption of proteins onto the substrate (Kasemo B, J. Prosth Dent. 49(6):832-837, 1983). The absorption takes place within the first 10 to 60 seconds of contact, long before the cells get access to the surface. This means that any cells which interact with the alloplast surface can only do so indirectly, through the absorbed protein layer.

The nature and amount of protein absorbed is specific to the alloplast composition (Uniyal S, Brash J L, Thromb. Haemost. 47, 285-290, 1982), depending on the physical and electromechanical properties of the given surface. It is conceivable that the absorbed protein contingent could determine what kind of cells interact with the alloplast surface (Bagambisa F B et al., Int. J. Oral Maxillof Implants 5, 217-226, 1994). Cell contact with the substrate is maintained by the formation of subcellular spatially and morphologically defined adhesion sites called focal adhesions. Focal adhesion are within 15 to 30 nm proximity of the substrate (Izzard C S, Lochner R L, J. Cell Sci. 21:129-159, 1976) and are about 2 to 10 μm long and 150 to 500 nm wide (Burridge K et al, Ann. Rev. Cell Biol., 487-525, 1988). Although the different phenomenological response of cells to material surfaces has been attributed to wetability, this can only be a first approximation (Parsegian V A, J. Of Prosth Dent. 49(6):838-841, 1983). It appears more useful to talk about the ability of the surfaces to interact with the key molecules involved in the orchestration of the post implantation interfacial events. If a material surface can not bind the macromolecules supportive of osteoblast function, the material is not likely to make a good bone implant. One way of getting bone cells to appose bone tissue onto the implant surface might be through having or creating surfaces that are attractant to the macromolecules responsible for events like cell phenomenology, growth and differentiation (Bagambisa F B et al. Int. J. Oral Maxillof. Implants 5:217-226, 1994).

The absorption onto Ti of aqueous solutions of matrix or matrix-like proteins has resulted in significant increases in the number of cells bound. This effect has been reported (Burridge K et al. Ann. Rev. Cell Biol. 487-525, 1988) and indicates that a specific cell receptormatrix protein interaction is a more efficient means of attachment than the undefined process of cell-Ti interaction.

Histological information is available on the interface between bone and implant material, but the understanding of the mechanisms operating when an implant is inserted into bone is limited and the concepts are speculative.

The process of integration is going on in an aqueous environment. When two bodies make contact, it is because they prefer each other to the intervening water or whatever else is originally between them. In the vicinity of an electrical charge, a molecule will turn to keep its attractive end close to the intruding charged body (Parsegian V A, J. Prosth Dent. 49(6):838-841, 1983). Small amounts of positively charged calcium ion will bind to certain electrically negative surface groups, displacing the water and replacing it with a bridge of (−) (+), (+) (−) configurations between bilayers. Expotentionally decay repulsion seen between bilayer membranes is seen also between single molecules (Parsegian V A, J. Prosth Dent. 49(6):838-841, 1983).

There are two paths in which a range of close interaction can be analyzed: first, the list of hydrogen bonds, hydrophobic bonds, salt bridges, van der Waals forces. Second, direct inspection of molecular contacts are they occur in protein monomers or tetramers the structures of which have been determined to atomic resolution by x-ray diffraction.

The metal surface is in fact a highly polarizable titanium oxide layer probably modified by accumulated impurities, from the bulk metal phase. With time, the titanium with oxide surface blends with material from adjacent tissue, and a thin layer of ground substance of cellular origin is deposited on the implant so as to cement bone tissue and titanium. The interactions of principal importance probably are electrostatic rather than van der Waals or hydrophobic interactions (Parsegian V A, J. Of Prosth Dent. 49(6):838-841, 1983). To a charged body, the highly polar oxide layer provides a strongly attractive alternative to water. The many configurations of titanium and oxygen likely to occur in such a surface provide a wide variety of adsorbant sites to attract various arrays of charge that probably reside on the water-soluble ground substance.

The oxide layer is so highly polar and therefore able to attract species that are ordinarily water soluble. Positive electrical charges in particular will move toward the oxide, for in addition to its polarizability the layer is negatively charged. It should not be surprising that such a highly polar region has been observed to incorporate (positive) calcium and (negative) phosphate ions from the adjacent aqueous phase. It is almost certain that the polar properties of adsorbant and substrate—not van der Waals forces, nor generalized electrical doubled layer, nor hydrophobic attractions—will determine contact (Parsegian V A, J. Of Prosth Dent. 49(6):838-841, 1983).

The chemical property of the titanium oxide surface suggests that calcium ions may be attracted to the oxide cover surface by electrostatic interaction with O— as just discussed. Calcium deposits have been observed in direct contact with the titanium oxide (Albrektsson T, and Hansson H A, Biomaterials, 7,201-205, 1986). According to the same model, calcium binding macromolecules may absorb selectively to the implant surface in vivo as the next sequence of events. Calcium binding molecules are often acidic with surface exposed carboxyl, phosphate or sulphate groups. Proteoglycans and/or proteins containing carboxyl and phosphate/sulphate groups may bind to the TiO₂ surface by this mechanism. Hydroxyapatite, the major mineral component of bone, also exhibits a surface dominated by negatively charged oxygen (P-bound) that can attract cations and subsequently anionic calcium binding macromolecules (Bernardi G and Kawasaki T, T: Chromatography of polypeptides and proteins on hydroxyapatite columns, Biochim. Biophys. Acta. 160, Pp 301-310, 1968). Glycosaminoglycans interact electrostatically with hydroxyapatite surface (Embery G and Rolia G, Interaction between sulphated macromolecules and hydroxyapatite studied by infrared spectroscopy. Acta Odontol. Scand, 38, 105-108, 1980). It has been shown that calcium absorbs to the surfaces after treatment with CaCl₂. The absorption of calcium onto the titanium implant surface when exposed to body fluids, increase its biocompatibility with bone and induce a subsequent adsorption of calcium binding macromolecules on to the implant surface. The surface characteristics of TiO₂ probably change from an anionic to a cationic state by the adsorption of calcium to the surface which will be subsequently have an increased ability to absorb acidic macromolecules like Opn. The results of the study were consistent with the proposal that calcium binding is a major mechanism by which proteins adsorb to TiO₂.

The present invention is further illustrated by the following non-limiting examples.

### EXAMPLE 1

### Coating of Implants

Titanium, plastic, glass and chromocobalt (CrCo) surfaces were coated with human recombinant OPN. Attachment and proliferation of human osteoblasts by means of matrix formation markers was evaluated using uncoated surfaces as a control. Also the amount of adhesion protein that can be coated to these surface was investigated.

The human recombinant phosphorylated form of osteopontin (rhOpn) was used as an adhesion molecule. This form of osteopontin migrates on 10% SD S-gels with an apparent molecular weight of 78 Kd, making it easy to differentiate from osteopontin secreted by osteoblasts which migrates in the same gels with an apparent molecular weight of 58 Kd.

The experiments outlined below investigate the expression and mineralization of extra cellular matrix components in human osteoblasts cultured on titanium disks, plastic, glass and chromocobalt surfaces coated with recombinant osteopontin. The adhesion molecule rhOPN used as a coating for these surfaces enhances attachment and proliferation of human osteoblasts cell lines, and increases the expression of matrix components when compared against uncoated surfaces.

Materials and Methods

Cell Culture of Human Osteoblasts

50,000 cells from the human osteoblastic cell line were seeded onto sterile titanium disks (11 mm in diameter) or titanium disks coated with recombinant Osteopontin placed inside a 24 well plate (12 mm diameter well) (Costar, Cambridge, Mass.). Cells were initially maintained in Dulbecco's Modified Medium (DME) supplemented with 10% fetal bovine serum until reaching confluence. The cells were then grown in DME media supplemented with 10% fetal bovine serum, 12.5 ug/ml ascorbic acid and 5 mM B-glycerophosphate (denoted as complete media).

Determination of Protein Absorption Onto Titanium Surfaces.

Titanium disks were cleaned in 10% Nitric acid for 12 hours, washed exhaustively with water, sterilized, then placed inside a 24 well plate (12 mm diameter well) (Costar, Cambridge, Mass.), and washed twice with 0.5 ml of sterile PBS. 0.1 milimolar CaCl₂ was added to 8 disks. Four different concentrations of the human recombinant osteopontin (60, 200, 400, 600 ug) were labeled with S35, and placed on all the titanium disks. After 24 hours, the bound and unbound protein was collected and counted using the Scintillation counter (Bergman 5000). The values among the two groups at the four concentrations were compared to determine the action of Calcium as a binding agent and the adequate concentration of the recombinant protein.

The Attachment of HOS Cells as a Function of the Substrate they were Grown on.

HOS cells were labeled overnight with 10 uCi ³H-thymidine, then dissociated from the plate with non-enzymatic dissociation solution (Sigma), washed 2 times with PBS, and counted. ³H-thymidine incorporated into TCA insoluble material was determined for the cells. 5000 cells (cpm total 1000) were plated onto coated or uncoated titanium disks and the disks incubated at 37° C. for 30 min. Unadhered cells were removed, and attached cells were washed 3 times with 0.5 ml PBS. The cells were lysed with ice cold 20% TCA and the radioactivity in the TCA insoluble fraction was determined using the Scintillation counter (Begman 5000).

The Proliferation of HOS Cells as a Function of the Substrate they were Grown on.

Cell proliferation was determined by the rate of ³H-Thymidine incorporation into DNA. Cells were labeled with 10 μCi/ml of ³H-Thymidine in DME media. After 6 hours, the cells were lysed in cold 10% trichloroacetic acid (TCA). The TCA insoluble material was collected and washed several times with 10% TCA, then resuspended in 0.5 N NaOH. ³H-thymidine incorporation into TCA insoluble material was used as an index of cell proliferation. The material collected was mixed with scintillation liquid (Begman). The amount of radiation generated was compared between cells grown in titanium disks uncoated, and titanium disks coated with OPN.

Synthesis of Osteopontin (Opn) and Bone Sialoprotein (BSP), and their Secretion and Deposition into the Extracellular Matrix.

Osteopontin and BSP were extracted from the extracellular matrix of HOS cells cultured on Ti disks or Ti disks coated with the recombinant Opn with lysis buffer (20 mM phosphate buffer, pH, 7.2, containing 150 mM NaCI, 0.1% SDS, 1 mM phenylmethylsulfonyl fluoride, 5 mM benzamidine, 0.1 mM e-amino caproic acid, 0.1 b-hydroxy mercuribenzoate, 0.1 mM pyrophosphate, 1 mM sodium fluoride, 1 mM sodium orthovanadate and 10 mM EDTA). Samples were then processed for Gel electrophoresis.

Western blot analysis: Cell layer proteins and conditioned media was electrophoresed in 10% SDS-polyacrylamide slab gels at 150 volts for 4 h. For Western blot analysis resolved proteins in gels were transferred by semi-dry blotting onto nitrocellulose membranes (Schleicher & Schuell, Keene, N H), gel transfers were carried out for 90 min. at 12 V in 0.025 M Tris-glycine buffer, pH 8.2, containing 20% methanol and 0.01% Tween 20 and 10% nonfat dry milk, then incubated with rabbit anti-mouse osteopontin (Ashkar S, et al., New York Academy of Science 760:296-298, 1995) in 20 mM Phosphate buffer, pH 7.4, containing 150 mM NaCL, 0.1% Tween 20 and 1% nonfat dry milk. After 1 h, the membranes were washed 3 times with 20 mM Phosphate buffer, pH 7.4, containing 150 mM NaCI, 0.1% Tween 20, then incubated with horseradish peroxidase-conjugated goat anti-rabbit 1 g antibodies for 1 h. Following several washing steps, the membranes were developed with ECL. Nonspecific interaction was assessed by the interaction of the primary and secondary antibodies with rabbit serum albumin.

Identification of proteins was made running the samples collected in a 7.5% SDS-polyacrylamide slab gels at 150 volts for 4 h. Then, the gels were stained by immersion in Coomassie blue for 24 hours. The gel was washed with 10% Acetic Acid, 20% Methanol, 70% ddWater, and the proteins identified by molecular weight against the standards ran with the samples.

The Expression of Alkaline Phosphatase Enzyme Activity on Human Osteoblast Cell Membranes in Culture.

Alkaline phosphatase enzyme activity was determined in glycine buffer pH 10.2 using p-nitrophenol phosphate as described (Gerstenfeld L C et al., Develop Biol; 122:4940, 1987). Briefly, cell layer was extracted with NP 40 (Detergent) in PBS for 10 min. at 4° C. 100 Iμl Aliquots were frozen until used. Then, the samples were thawed and prepare in glycine buffer plus p-nitrophenol phosphate for one hour at 37° C. After the samples turned yellow, the reaction was stopped with 0.2 milimolar Na OH, and the samples were read in the spechtometer (Begmann).

Determination of Mineral Content of Human Osteoblast Cell Culture.

HOS cells were grown either on coated or uncoated titanium disks. Media was supplemented with ascorbate and b-glycerol phosphate to stimulate the mineralization of the extracellular matrix. After two weeks, media was removed and the cells were lysed with triton. Then, all soluble components were removed and calcium content was determined using quantitative, calorimetric determination at 575 μm (Sigma Diagnostics Calcium). Basically, calcium reacts with o-cresolphthalein, a chromogenic agent that in an alkaline medium forms a purple colored complex. The intensity of the color, measured at 573 nm, is directly proportional to calcium concentration in the sample.

Determination of Peptides Exhibiting Binding and Cell Spread Activity

Example 11 analyzes the binding and cell spread activity of various peptides. The activities were measured in 24-well plates that were coated overnight at 4° C. with 10 μg/ml of the indicated ligand or peptide and then blocked for one hour at room temperature with 10 mg/ml BSA (Bovine Serum Albumin) in PBS (Phosphate Buffered Saline). To preserve the integrity of the adhesion receptors osteoprogenitor cells were harvested from sub-confluent cultures by non-enzymatic cell dissociation solution (Sigma, St. Louis, Mo.). Cells were washed twice with PBS and re-suspended at a concentration of 1×10⁵ cells/ml of sterile Ca²⁺- and Mg²⁺-free PBS, supplemented with 0.1% BSA and 1 mM sodium pyruvate. 5×10⁴ cells were incubated in each well and, after one hour at 37° C., the wells were washed three times with 0.5 ml PBS to remove non-adherent cells, fixed in 1% glutaraldehyde in 0.1 M cacodylate buffer (pH 7.4) at room temperature for one hour then stained with toluidine blue and hematoxylin. The total number of attached or spread cells in each well were counted microscopically using a Nikon Eclipse microscope equipped with a Sony digital camera. Total number of attached or spread cells were quantified using an Optima 5.2 image analysis system. Each experiment was done in triplicate and is reported as mean+/−standard error. To minimize variability inherent to cell attachment studies cells were scored as attached only when a defined nucleus observed accompanied by a transition from round to cuboidal cell morphology. Round cells that are loosely attached with no defined nucleus were scored as non-attached. These cells can be removed with repeated washes. The viability of the cells was measured before and after the termination of the experiments and only data from experiments with greater than 95% cell viability were used. Further, under the conditions used in these experiments, cell attachment was temperature dependent, inhabitable by trypsin treatment and not affected by inhibitors of protein synthesis or secretion. Cell spreading was determined by membrane contour analysis and was scored according to increase in cell volume/surface area. Because of this change in cell volume/surface area, cell spreading is a measure of a change in cellular development. In some experiments, cell spreading was also assessed by the formation of stress fibers. The formation of stress fibers and/or changes in cell volume/surface area are each characteristics of cells undergoing differentiation. Each experiment was performed in quadruplicate wells and repeated three times.

Chemotaxis

Directed migration of cells (chemotaxis) are determined in multi-well chemotaxis chambers as described. (Weber et al. (1996) Science 26:271:509-512). Briefly, two-well culture plates (Transwell) with polycarbonate filters (pore size 8-12 μm) separating top and bottom wells were coated with 5 μg fibronectin. 2×10⁵ cells are added to the upper chamber and incubated at 37° C. in the presence or absence of the peptide of interest in the lower chamber. After 4 h, the filters are removed, fixed in methanol, stained with hematoxylin and eosin and cells that migrate to various areas of the lower surface are counted microscopically. Controls for chemokinesis include 200 ng of osteopontin in the top well. All assays are done in triplicate and reported as mean+/−standard deviation.

Haptotaxis

Haptotaxis of cell lines to peptides or fragments are assayed using a Boyden chamber. The lower surface or both sides of polycarbonate filters with 8 μm pore size were coated with different amounts of peptide. 2×10⁵ cells are added to the upper chamber, and incubated at 37° C. in the absence of any factors in the lower chamber. After 4 h the filters are removed, fixed in methanol and stained with hematoxylin and eosin. Cells that migrate to the lower surface are counted under a microscope. All assays are done in triplicate and are reported as a mean+/−standared deviation.

### EXAMPLE 2

### Effect of Ca++ Ions on the Binding of Osteopontin to Ti Disks

Increasing concentration of ³⁵S-labeled OPN (60, 200, 400, 600 ug) were incubated with titanium disks either with (▪) or without ( [symbol] ) CaCl₂ at 4° C. After 24 h the unbound protein was removed and the Ti disks were washed with PBS. Bound OPN was extracted from the disks with scintilation fluid and counted. Each experiment was done in triplicates and reported as mean±SEM.

To investigate whether exogenously added Ca++ had any effect on the binding of rhOPN to Ti, the binding of rhOPN to Ti disks was measured with and without added CaCl₂. The results, presented in FIG. 1, demonstrate that in the absence of added CaCl₂ the Ti disks saturate at 60 μg of rhOPN, but in the presence of 100 mM CaCl₂ the Ti disks can bind more rhOPN saturating at more than 110 μg protein/disks.

### EXAMPLE 3

### Attachment of HOS Cells to Ti Surfaces Coated with rhOPN

5000 cells (total cpm 1000) were plated on either coated or uncoated Ti disks and incubated at 37° C. in a humidified atmosphere (95% air 5% CO₂). After 30 min, unattached cells were removed and the disks were washed with PBS. The total number of attached cells was determined for the total cpm released for the disks after the cells were lysed with 10% TCA and solubilized in 5 ml scintillation fluid. All measurements were done in triplicates and graphed as mean±Standard error of the mean.

The initial events following seeding of cells onto Ti surfaces include the attachment, migration and proliferation of the seeded cells. Coating Ti disks with 50 μg of rhOPN enhanced by 1100% the attachment of HOS cells to Ti disks (FIG. 2), after 30 min. These results are consistent with the role of osteopontin in promoting cell attachment and spreading.

### EXAMPLE 4

### Proliferation of HOS Cells on Ti Surfaces Coated with Phosphorylated Human Recombinant Opn

Cell proliferation was determined by the rate of ³H-Thymidine incorporation into DNA. Cells labeled with ³H-Thymidine were seeded for 6 hours, then lysed with TCA. The TCA insoluble material was collected and resuspended in 0.5 N NaOH. ³H-thymidine incorporation into TCA insoluble material was used as an index for cell proliferation. Rate of proliferation is expressed as cpm/1000 cells/6 h. Control group: 254,54, rhOPN group: 560,83. All measurements were done in triplicates and reported as mean±Standard error for the mean.

Since rhOPN promoted cell attachment to Ti disks, it was of interest to examine whether the protein had any effect on the proliferation of HOS grown on Ti disks. Measurement of the rate of proliferation of HOS cells grown on coated or uncoated Ti disks showed that the proliferation rate of cells grown on rhOPN coated Ti disks was approximately twice (FIG. 3) the proliferation rate of cells grown on uncoated Ti disks.

### EXAMPLE 5

### Secretion of Osteopontin and BSP by HOS Cells Growing on Coated Ti Disks

Cell layer proteins and conditioned media was electrophoresed in 10% SDS-polyacrylamide slab gels at 150 volts for 4 h. The resolved proteins were transferred by semi-dry blotting onto nitrocellulose membranes for 90 min. at 12 V in Transfer Buffer. Then, the membranes were incubated with either rabbit anti-mouse osteopontin or rabbit anti-mouse BSP. After 1 h, the membranes were washed 3 times with PBST. Then incubated with horseradish peroxidase-conjugated goat anti-rabbit Ig antibodies for 1 h. Following several washing steps in PBST, the membranes were developed with ECL as described by the manufacturer (Amersham, London).

Osteopontin and BSP were extracted from the extracellular matrix of HOS cells cultured on Ti disks or Ti disks coated with the recombinant Opn with lysis buffer. Samples were then processed for Gel electrophoresis. Western blot analysis for OPN secretion into the extracellular matrix showed increased secretion of OPN from cells grown on coated Ti disks when compared to cells grown on uncoated titanium controls as denoted. Assays for Opn expression by Western blot were done by triplicate.

BSP extracellular matrix secretion expressed by Western blot analysis was less marked than the production of osteopontin from cells grown on the rhOPN coated implants. Cells in the control groups did not expressed bone sialoprotein. Assays for BSP expression by Western blot were done by triplicate.

### EXAMPLE 6

### Expression of Alkaline Phosphatase Enzyme Activity on Human Osteoblast Cell Membranes in Culture

Alkaline phosphatase enzyme activity was determined in glycine buffer pH 10.2 using pnitrophenol phosphate. Cell layer was extracted with NP 40 in PBS for 10 min. at 4° C. 100 μl Aliquots were used. The alkaline phosphatase activity determined by colorimetric assay (as described in materials and method). A unit is defined as the amount of enzyme which releases 1 μmol of p-nitrophenol/h. All measurements were done in triplicates and reported as mean±Standard error of the mean.

Since secreted proteins and extracellular matrix production was different between cells grown on coated and uncoated disks, the levels of alkaline phosphatase in both groups were examined to assess the extent of differentiation of HOS cells grown on coated Ti Surfaces. The results presented in FIG. 4, indicate that the levels of alkaline phosphatase activity in cells grown on Ti disks decreased over the levels of Apase detected in the control groups. These results are consistent with the observations that Apase activity decreases as osteoblasts differentiate into mature matrix producing cells.

### EXAMPLE 7

### Extracellular Matrix Mineralization of HOS Cells Grown on Either Coated or Uncoated Ti

HOS cells were grown either on coated or uncoated titanium disks. Media was supplemented with ascorbate and β-glycerol phosphate. After two weeks, media was removed and the cells were lysed. Then, all soluble components were removed and calcium content was determined using quantitative, colorimetric determination at 575 nm (Sigma Diagnostics Calcium). All measurements were done in triplicates and reported as mean±Standard error in the mean.

When cultured in the presence of ascorbate and β-glycerol phosphate, HOS cells grown on coated Ti disks mineralized their extracellular matrix within 2 weeks (FIG. 5) in a manner similar to HOS cells cultured on plastic. However, HOS cells grown on uncoated Ti disks under similar conditions did not mineralize their extracellular matrix. These results and the results presented above suggest that when cultured on uncoated Ti disks HOS cells attach, proliferate and differentiate at a slower rate than when cultured on coated disks. Furthermore, HOS cultured on coated disks synthesize an extracellular matrix that mineralizes within two weeks. In several respects HOS cells grown on Ti surfaces coated with rhOPN develop in a manner similar to cells grown on plastic dishes.

### EXAMPLE 8

### Attachment of HOS Cells to Surfaces Coated with OPN

500 cells were plated on coated plastic, glass or chromocobalt surfaces and incubated at 37° C. in a humidified atmosphere (95% air 5% CO₂). Surfaces were coated with either human recombinant phosphorylated OPN (rhOPN) or unphosphorylated OPN. Fibronectin coated surfaces were used as a control. After 1 hour, unattached cells were removed and the surfaces were washed with PBS. The total number of attached cells was determined for the total cpm released for the surfaces after the cells were lysed with 10% TCA and solubilized in 5 ml scintillation fluid. All measurements were done in triplicates. The results are outlined in Table 2 below.

TABLE 2
Attachment of HOS Cells to OPN coating.

| Surface | % total attached |
|---|---|
| Plastic | |
| OPN | 43.6 |
| OPN-p | 90.8 |
| Fibronectin | 91.6 |
| glass | |
| OPN | 37.2 |
| OPN-p | 98.1 |
| fibronectin | 89.6 |
| chromocobalt (CrCo) | |
| OPN | 4 |
| OPN-p | 69.2 |
| fibronectin | 54.8 |

OPN = unphosphorylated OPN
OPN-p = phosphorylated OPN

The results outlined above demonstrate that human recombinant phosphorylated OPN (rhOPN) promoted cell attachment at the same or higher rate then fibronectin. These results are consistent with the role of osteopontin in promoting cell attachment and spreading.

### EXAMPLE 9

### Proliferation of HOS Cells on Surfaces Coated with Phosphorylated Human Recombinant Opn

Cell proliferation was determined by the rate of ³H-Thymidine incorporation into DNA. Cells labeled with ³H-Thymidine were seeded for 6 hours, then lysed with TCA. The TCA insoluble material was collected and resuspended in 0.5 N NaOH. ³H-thymidine incorporation into TCA insoluble material was used as an index for cell proliferation. Rate of proliferation is expressed as cpm/1000 cells/6 h. All measurements were done in triplicates.

Since rhOPN promoted cell attachment to different surfaces, it was of interest to examine whether the protein had any effect on the proliferation of HOS grown on these surfaces. Measurement of the rate of proliferation of HOS cells grown on coated or uncoated glass, plastic and chromocobalt surfaces showed that the proliferation rate of cells grown on rhOPN coated surfaces was at least twice (Table 3) the proliferation rate of cells grown on uncoated surfaces.

TABLE 3
Proliferation of HOS Cells on OPN

| | Proliferation Rate |
|---|---|
| Surface | (Rate Cpm/6 h/1000 cells) |
| Plastic only | 1100 |
| Plastic + rhOPN | 3300 |
| Glass only | 310 |
| Glass + rhOPN | 2740 |
| CrCo only | 120 |
| CrCo + rhOPN | 1740 |

### EXAMPLE 10

### In Vivo Studies of Ti Coated rhOPN Implants

Forty implants (5 per quadrant) were placed in four Haundel/Labrador dogs after extraction of four premolars (PM1-PM4) and one molar (M1), and a three month healing period. Eight hollow screw Ti implants were coated with rhOPN. Eight uncoated implants served as controls. The remaining implants were coated with 3 additional different molecules denoted as study 2, study 3, and study 4.

Prior to implant placement, core samples from the donor place were taken to histologically analyze bone quality after extractions. This procedure, also ensured a hollow space for bone ingrowth inside the coated and uncoated implants. Dogs were sacrificed after 4 and 8 weeks.

Implants were recovered for histological analysis. Each implant was sectioned vertically. The core inside the hallow implant was removed using liquid nitrogen. Decalcified sections were embedded in paraffin and stained using Herovichi's techniques to differentiate immature from mature collagen. Light microscopy at 4× and 40× magnifications were used to compare histological differences between rhOPN coated implants and uncoated implants.

The in vivo results show enhanced bone healing around coated implants. Uncoated implants show normal bone healing characterized by granulation tissue and a few areas of vascularization and matrix deposition after four weeks. These results demonstrate that coating titanium implants with rhOPN reduces healing time around dental implants.

### EXAMPLE 11

### In Vivo Studies of Implants Coated with Recombinant Osteopontin (rOPN) or mOC-1016 (SEQ ID NO:15)

Since titanium does not induce bone formation, one way of assuring the apposition of bone cells to the implant is to design an implant surface that is attractant to these molecules. The present Example evaluates the enhancement of osseointegration of dental implants coated with either osteopontin or mOC 1016 (SEQ ID NO:15) as compared to non-coated titanium plasma sprayed (TPS) surface in a canine model. Canine models allow the use of implant designs and sizes commonly used in clinical applications.

Mandibular premolars and first molars were extracted bilaterally in a total of twelve dogs. After a healing period of three months, 6 implants TPS (ITI 4.1×8 mm) were placed in each mandibular quadrant. Test coatings of rOPN and mOC-1016 and non-coated TPS control wre used. A randomized distrubution of tests and controls were incorporated. Test solutions were comprised of mannitol, sucrose, citric acid, water and OPN and OC-1016.

Large hounds were used in this Example. For all surgical procedures (extractions, implant placement, suture removal), after sedation with acepromazine (0.1 mg/kgS.C.), the animals were anesthetized using pentobarbitol (20 mg/kg I.V.). Antibiotics (bicillin 200,000 u I.M.) and ibuprofen for pain control (200 mg/dog/day P.O.) were given for extraction and implant placement surgeries. For sacrifice, an overdose of pentobarbitol (50 mg/kg I.V. after sedation with acepromazine 0.1 mg/kg S.C.) was administered.

The implant and it's surrounding tissue were dissected in block, fixed in 4% formaldehyde and imbedded in PMMA. The osseointegration of the implant was measured as percent of directly apposed bone versus total implant area by contour analysis using an image analysis system. The extent of new mineralized bone (in percent) in the area between the major and minor diameter of the implant was also determined.

Assessment of the bone to implant contact (BIC) at 4 weeks indicated that the implants treated with rOPN or mOC-1016 had significantly greater BIC than non-coated implants (Table 4). Mean percent bone to implant contact was 45.00, 65.6, 61.3, 64.9, 73.1, and 70.3% in the non-coated implants, rOPN at 200 μg/ml, and mOC-1016 25, 50, 100 and 200 μg/ml groups respectively. At the 12 week time point, the treatment groups again had greater bone to implant contact when compared to non-coated implants (Table 5). Mean percent bone to implant contact was 46.5, 62.5, 58.3, 54.7, 60.5, and 59.7% in the non-coated implants, rOPN 200 μg/ml, and mOC-1016 25, 50, 100 and 200 μg/ml groups respectively. The rOPN 200 μg/ml had statistically significant greater BIC when comparing to uncoated implants at 12 weeks.

The bone density surrounding the implants at 4 weeks post-implantation have been summarized in Table 6. Mean percent bone density was 54.5, 70.9, 67.8, 69.3, 76.9, and 72.6% in the non-coated implants, rOPN at 200 μg/ml, and mOC-1016 25, 50, 100 and 200 μg/ml groups respectively. The difference between the response of all treatment were statistically significantly greater than the non-coated implant group. The 12-week bone density results indicate a statistically significant difference among the treatment groups (Table 7).

The in vivo results outlined above indicate that OPN and mOC-1016 are able to accelerated the healing of dental implants in the canine model. Bone to implant contact percentages were statistically greater in the treatment groups compared to the untreated implants at 4 weeks. The rOPN and mOC 1016 treated dental implants were able to improve the density of bone as compared to untreated implants. Bone density percentages were statistically greater in the treatment groups when compared to the untreated implants at 4 weeks. At 12 weeks post-implantation, OPN and mOC-1016 treated implants were significantly different when compared to non-coated implants.

The issue of safety was also addressed by assessing the serum chemistry, hematology and antibody results following treatment. These safety assessments indicate that there are no clinically significant differences from baseline. Therefore, rOPN and OC-1016 are safe based upon these evaluations.

The results outlined above also demonstrate that coating of different surfaces, e.g., titanium disks, glass, plastic, or CrCo, with phosphorylated human recombinant osteopontin enhances the rate of attachment and proliferation of human osteoblast cell lines in vitro when compared to uncoated surfaces. This enhancement is demonstrated by better attachment and proliferation of the cells, increased production of the extracellular matrix components, and its faster calcification. These results also contribute to the understanding of the molecular events that may be occurring in the healing of bone around the implants.

TABLE 4
Mean Percent Bone to Implant Contact at 4 Weeks

| | Mean | SEM | SD |
|---|---|---|---|
| TPS | 45.0 | 3.5 | 12.1 |
| OPN 200 μg/ml | 65.6 | 2.8 | 9.6 |
| OC-1016 25 μg/ml | 61.3 | 3.0 | 10.3 |
| OC-1016 50 μg/ml | 64.9 | 3.3 | 11.4 |
| OC-1016 100 μg/ml | 73.1 | 3.1 | 10.8 |
| OC-1016 200 μg/ml | 70.3 | 2.9 | 10.2 |

TABLE 5
Mean Percent Bone to Implant Contact at 12 Weeks

| | Mean | SEM | SD |
|---|---|---|---|
| TPS | 46.5 | 1.7 | 5.5 |
| OPN 200 μg/ml | 62.5 | 3.2 | 10.1 |
| mOC-1016 25 μg/ml | 58.3 | 2.9 | 9.2 |
| mOC-1016 50 μg/ml | 54.7 | 5.0 | 15.7 |
| mOC-1016 100 μg/ml | 60.5 | 3.6 | 10.7 |
| mOC-1016 200 μg/ml | 59.7 | 2.9 | 9.1 |

TABLE 6
Mean Percent Bone Density at 4 weeks

| | Mean | SEM | SD |
|---|---|---|---|
| TPS | 54.5 | 2.5 | 8.6 |
| OPN 200 μg/ml | 70.5 | 3.7 | 12.9 |
| mOC-1016 25 μg/ml | 67.9 | 4.8 | 16.5 |
| mOC-1016 50 μg/ml | 69.3 | 3.4 | 11.7 |
| mOC-1016 100 μg/ml | 76.9 | 3.0 | 10.3 |
| mOC-1016 200 μg/ml | 72.6 | 3.5 | 12.1 |

TABLE 7
Mean Percent Bone Density at 12 Weeks

| | Mean | SEM | SD |
|---|---|---|---|
| TPS | 58.7 | 3.5 | 11.1 |
| OPN 200 μg/ml | 56.9 | 5.4 | 17.0 |
| MOC-1016 25 μg/ml | 65.8 | 3.8 | 12.1 |
| MOC-1016 50 μg/ml | 56.9 | 5.1 | 16.1 |
| MOC-1016 100 μg/ml | 63.5 | 4.4 | 13.2 |
| MOC-1016 200 μg/ml | 57.9 | 3.0 | 9.6 |

### EXAMPLE 12

### Peptide Binding and Cell Spread

5000 total human osteoprogenitor cells were plated on either uncoated plates or coated and incubated at 37° C. in a humidified atmosphere (95% air, 5% CO₂). After 30 minutes, unattached cells were removed and the plates were washed with PBS. The total number of attached cells was determined as described in the materials and methods. In some experiments (labeled with an “#” in Table 8), antibodies (0.1 g/ml) against various integrins were incubated with the cells for 15 minutes prior to plating.

When coated with osteopontin, mOC-1016 (SEQ ID NO:15), SEQ ID NO:9, SEQ ID NO:10, SEQ ID NO:11, SEQ ID NO:12, SEQ ID NO:13, or SEQ ID NO:14, plates coated with human osteoprogenitor cells undergo a transformation from a neutral (uncoated condition) to a proactive condition in which the number of attached cells, as well as the percent spread, significantly increases (Table 8). Table 8 also illustrates that antibodies to different integrins may be used to block binding to specific integrins. For example antibodies to αᵥβ3 integrin significantly diminish mOC-1016 binding (See Table 8). Such antibodies may be used to abolish or attenuate the activity of specific peptides, like OC-1016, in vivo.

Table 8 also provides evidence that peptides mOC-1016 (SEQ ID NO:15), SEQ ID NO:9, SEQ ID NO:10, SEQ ID NO:11, SEQ ID NO:12, SEQ ID NO:13, and SEQ ID NO:14, each bind to osteoprogenitor cells and significantly increase cellular attachment over the control. These same peptides also regulate cellular development of human osteoprogenitor cells by increasing the percentage of “cell spread”. As noted in the material and methods, cell spread is measured by the change in cell volume to surface area, as well as the formation of stress fibers. Changes in morphological characteristics such as these, indicate that the cell is undergoing significant genetic and biochemical changes and being directed to the next step in the developmental pathway towards a differentiated phenotype.

TABLE 8
Peptide binding and cell attachment.

| | % total attached cells | STD | % Spread | STD |
|---|---|---|---|---|
| Control | 43.6 | 6.54 | 8.4 | 0.924 |
| Osteopontin | 78.4 | 11.76 | 78.4 | 9.408 |
| SEQ ID NO: 15 | 91.6 | 13.74 | 84.4 | 16.88 |
| # Anti-αᵥβ3 | | | | |
| Control | 31.2 | 4.68 | 5.2 | 0.52 |
| Osteopontin | 69.2 | 10.38 | 24.8 | 4.464 |
| SEQ ID NO: 15 | 48 | 7.2 | 12.4 | 1.24 |
| # Anti-CD44 | | | | |
| Control | 18.8 | 2.82 | 6.8 | 1.02 |
| Osteopontin | 66 | 9.9 | 46.4 | 7.888 |
| SEQ ID NO: 15 | 96.4 | 14.46 | 86.8 | 13.02 |
| # Anti-αβ1 | | | | |
| Control | 23.7 | 3.792 | 7.1 | 1.065 |
| Osteopontin | 74.8 | 11.968 | 56.6 | 8.49 |
| SEQ ID NO: 15 | 96.8 | 15.488 | 89.3 | 13.395 |
| Control | 22.6 | 4.17 | 3.0 | 1.5 |
| SEQ ID NO: 9 | 66.6 | 12.8 | 54.4 | 6.45 |
| Control | 22.6 | 4.17 | 3.0 | 1.5 |
| SEQ ID NO: 10 | 98.8 | 21.7 | 77.9 | 13.2 |
| Control | 22.6 | 4.17 | 3.0 | 1.5 |
| SEQ ID NO: 11 | 84.6 | 9.7 | 91.6 | 6.4 |
| Control | 22.6 | 4.17 | 3.0 | 1.5 |
| SEQ ID NO: 12 | 73.9 | 12.2 | 88.6 | 13.7 |
| Control | 22.6 | 4.17 | 3.0 | 1.5 |
| SEQ ID NO: 13 | 91.1 | 20.6 | 100.0 | 2.8 |
| Control | 22.6 | 4.17 | 3.0 | 1.5 |
| SEQ ID NO: 14 | 90.0 | 9.7 | 99.2 | 11.5 |

### EXAMPLE 12

### C-terminus of Osteopontin Binds Eosinphils

White blood cells are grouped into three major categories, granulocytes, monocytes, and lymphocytes.

Lysosomes and secretory vesicles are the main components of granulocytes. Based on the morphology and staining properties of these organelles, granulocytes are subdivided into three more categories, neutrophils, which serve to phagocytose bacteria; basophils, which aid in inflammatory reactions by secreting histamine, and eosinophils, which destroy small organisms and mediate allergic inflammatory responses.

Selective binding data has revealed that peptides comprising the C-terminal 59 amino acids of osteopontin are able to bind eosinophils in vitro. The sequence is identified as EHSDVIDSQELSKVSREFHSHEFHSHEDMLVVDPKSKEEDKHLKFRIS HELD SASSEVN (SEQ ID NO:16). The binding data has been obtained using the cell attachment and spreading assays described above in the Material and Methods.

One will notice that the peptide does not contain an “RGD” sequence. As discussed above, interactions via integrins and other receptors with the extracellular matrix are critical to the infiltration and accumulation of eosinophils in many diseases. Peptides lacking an RGD sequence yet harboring further sequence similarities to the amino acid sequence of SEQ ID NO:15 are of particular interest because such peptides contradict previous evidence suggesting that the RGD sequence is necessary for binding to integrin receptors. Peptides such as these may be used to develop therapies for the treatment of such eosinophil-associated diseases listed above. For example, the peptide may be modified such that binding to the cell inhibits interaction with extra-cellular matrix (ECM) components and therefore migration.

### EXAMPLE 13

### Antibodies to SEQ ID NO:8 (OC 1016) and SEQ ID NO:15 (mOC-1016)

Antibodies are very selective proteins that are able to bind a single target among many. A major limitation in the therapeutic use of a particular antibody is producing it in a large enough quantity to be useful. Advances in monoclonal antibody technology have provided a route to be used to secure significant amounts of these useful diagnostic and therapeutic proteins. The antigen of interest is injected into a mouse in order to elicit an immune response. Lymphocytes, another type of white blood cell that produce antibodies, are stored in the spleen of the animal. The spleen is removed and the cells are fused to a specialized myeloma cell line. The fused cells (hybridomas) now produce antibodies specified by the lymphocytes from the immunized animal. These hybridomas also retain characteristics of myeloma cells, in that they continue to grow and divide in culture, producing a relatively unending supply of antibody.

This monoclonal antibody technology has allowed for the production of antibodies against OC-1016 and modified OC-1016. Two hybridoma cell lines have been produced to generate antibody against OC-1016 and modified OC-1016 and are denoted herein as HYB 1016(a) and HYB 1016(b).

### EXAMPLE 14

### Regulating Osteopontin Expression

Additionally, two more cell lines have been established in which human osteopontin is under the control of a CMV (cytomegalo virus) promoter. These cell lines are human osteoblasts (Table V). Methods used to generate these cell lines are familiar to one of skill in the art and can be employed using conventional techniques of cell biology, cell culture, molecular biology, transgenic biology, microbiology, recombinant DNA, and immunology. The literature is replete with such techniques. See, for example, Molecular Cloning A Laboratory Manual, 2^{nd} Ed., by Sambrook, Fritsch, and Maniatis (Cold Spring Harbor Laboratory Press: 1989); DNA Cloning, Volumes I and II (D. N. Glover ed., 1985); Handbook of Experimental Immunology, Volumes I-1 (D. M. Weir and C. C. Blackwell, eds., 1986); and Manipulating the Mouse Embryo, (Cold Spring Harbor Laboratory Press, Cold Spring Harbor, N.Y., 1986).

The cell lines presented here may be used to regulate the expression of human osteopontin. The expression of osteopontin in vivo varies and its presence in migrating fibroblastic cells complicates an understanding of it's expression pattern even further. Zohar et al., 1997, Cell Physiol. 170:88-100.

It is clear that osteopontin plays an indirect yet significant role in regulating the expression of developmentally regulated genes. By providing an osteoblast cell line in which the expression of osteopontin at a high level and constitutive, one is able to determine the effects of this expression of a developmentally critical ligand on the development of, for example, osteoblasts into osteocytes and lining cells. Once this is determined, therapies are developed to address issues associated with bone remodeling and periodontal disease. One is able to further regulate cellular differentiation/development in vivo by expressing osteopontin that is produced and secreted by neighboring cells. Examples of cells harboring such an osteopontin expressible construct are osteoprogenitor cells, tumor cells, macrophages, periosteal cells, endothelial cells, epithelial cells, eosinophils, stem cells, osteoblasts, osteocytes, cementoblasts, fibroblasts, limited potential precursor cells, precursor cells, committed precursor cells, and differentiated cells.

One of skill in the art will recognize that the expression of osteopontin is not limited to constitutive expression driven by a CMV promoter. The expression of osteopontin may also be regulated using nucleic acid constructs harboring any number of available heterologous regulatable promoters. Heterologous is used herein to describe any promoter other than the native osteopontin promoter. The two cell lines described herein are referred to as HOBOP1 (Clone 1a—Human osteoblasts expressing hOPN1a under the control of CMV promoter) and HOBOP1 (Clone 2a—Human osteoblasts expressing hOPN1a under the control of CMV promoter).

Equivalents

Those skilled in the art will be able to recognize, or be able to ascertain using no more than routine experimentation, numerous equivalents to the specific procedures described herein. Such equivalents are considered to be within the scope of this invention and are covered by the following claims.

1. An active osteopontin peptide fragment comprising an amino acid sequence selected from the group consisting of SEQ ID NO:7, SEQ ID NO:8, SEQ ID NO:9, SEQ ID NO:10. SEQ ID NO:11, SEQ ID NO:12, SEQ ID NO:13, SEQ ID NO:14, and SEQ ID NO:15, wherein the peptide binds to at least one integrin receptor on a cell surface selected from the group consisting of αᵥβ₃, αᵥβ₅, 4β₁, 2β₁, VCAM, ICAM CD44, V₃Vₓ.

2. The peptide fragment of claim 1, wherein the peptide increases cell attachment to a biomaterial and increases cell spread.

3. The peptide fragment of claim 1, wherein the peptide binds to at least one integrin receptor on a cell surface selected from the group consisting of VCAM, ICAM CD44, and V₃Vₓ.

4. (canceled)

5. The peptide fragment of claim 1 wherein the integrin(s) is selected from the group consisting of αᵥβ₃, αᵥβ₅, 4β₁, and 2β₁.

6. The peptide fragment of claim 1 wherein the cell is selected from the group consisting of osteoprogenitor cells, tumor cells, macrophages, periosteal cells, endothelial cells, epithelial cells, eosinophils, stem cells, limited potential precursor cells, precursor cells, committed precursor cells, and differentiated cells.

7-18. (canceled)


SEQUENCE LISTING (16 sequences)

SEQ ID NO: 1 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 2 - protein, 6 aa, Artificial Sequence
  Osteopontin-derived peptide
LVLDPK

SEQ ID NO: 3 - protein, 6 aa, Artificial Sequence
  Osteopontin fragment
LVVDPK

SEQ ID NO: 4 - protein, 5 aa, Artificial Sequence
  Artificial peptide sequence
RGRDS

SEQ ID NO: 5 - protein, 5 aa, Artificial Sequence
  Artificial peptide sequence
GRGDS

SEQ ID NO: 6 - protein, 32 aa, Artificial Sequence
  Artificial peptide sequence
VFTPVVPTVDTYDGRGDSVVYGLRSKSKKFRR

SEQ ID NO: 7 - protein, 33 aa, Artificial Sequence
  Artificial peptide sequence
VFTPVVPTVDTYDGRGDSVVYGLRSKSKKFRRP

SEQ ID NO: 8 - protein, 40 aa, Artificial Sequence
  Artificial peptide sequence
RSRRATEVFTPVVPTVDTYDGRGDSVVYGLRSKSKKFRRP

SEQ ID NO: 9 - protein, 49 aa, Artificial Sequence
  Artificial peptide sequence
SDELVTDFPTDLPATEVFTPVVPTVDTYDGRGDSVVYGLRSKSKKFRRP

SEQ ID NO: 10 - protein, 40 aa, Artificial Sequence
  Artificial peptide sequence
RSRRATEVFTPVVPTVDTYDGRGDSVVYGRRSKSKKFRRP

SEQ ID NO: 11 - protein, 63 aa, Artificial Sequence
  Artificial peptide sequence
RSRRATEVFTPVVPTVDTYDGRGDSVVYGRRSKSKKFRRPAGAAGGPAGPAGPAGPAGPAGPA

SEQ ID NO: 12 - protein, 36 aa, Artificial Sequence
  Artificial peptide sequence
RSRRVFTPFIPTESANDGRGDSVAYGLKSKSKKFRR

SEQ ID NO: 13 - protein, 32 aa, Artificial Sequence
  Artificial peptide sequence
DTFTPIVPTVDVPNGRFDSLAYGLKSKSKKFQ

SEQ ID NO: 14 - protein, 40 aa, Artificial Sequence
  Artificial peptide sequence
RSRRATEVFTPVVPTVDTYDGRADSVVYGRRSKSKKFRRP

SEQ ID NO: 15 - protein, 40 aa, Artificial Sequence; identical to SEQ ID NO: 8
  Artificial peptide sequence

SEQ ID NO: 16 - protein, 59 aa, Artificial sequence
  Osteopontin fragment
EHSDVIDSQELSKVSREFHSHEFHSHEDMLVVDPKSKEEDKHLKFRISHELDSASSEVN